(19) United States
(12) Patent Application Publication
(10) Pub. No.: US 2011/0033463 A1
(43) Pub. Date: Feb. 10, 2011

(54) APHERESIS, ADMINISTRATION OF AGENT, OR COMBINATION THEREOF

(75) Inventors: Deepak R. Thakker, Blaine, MN (US); Lisa L. Shafer, Stillwater, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/852,170

(22) Filed: Aug. 6, 2010

Related U.S. Application Data
(60) Provisional application No. 61/231,782, filed on Aug. 6, 2009.
(60) Provisional application No. 61/231,784, filed on Aug. 6, 2009.

(51) Int. Cl. A61K 39/395; A61K 38/28; A61K 31/192; A61K 31/439; A61K 31/4439; A61K 38/18; A61K 38/48; A61P 25/28; A61P 25/00; A61K 31/7105; A61K 31/4406; A61K 31/55

(52) U.S. Cl. 424/134.1; 424/172.1; 514/5.9; 514/570; 514/305; 514/342; 514/8.1; 514/8.6; 424/94.67; 424/94.63; 514/17.7; 514/7.6; 514/569; 514/44 A; 424/133.1; 514/357; 514/340; 514/212.04

(57) ABSTRACT

A device is configured to remove a target molecule from a bodily fluid of a subject and to deliver a therapeutic agent to the subject. Such a device may be used for treatment of a disease associated with amyloid beta accumulation in the subject. Agents selected from the group consisting of an ApoE-modulating agent; a RAGE inhibitor; a β-secretase 1 (BACE1) inhibitor; a γ-secretase inhibitor; a muscarinic receptor subtype 1 (M1) agonists; a growth factor; an enzyme capable of degrading amyloid beta; a mitochondrial antioxidant; insulin; and an inhibitor of tumor necrosis factor (TNF) may be administered directly to the central nervous system of a subject for treatment of a disease associated with amyloid beta accumulation.

### CROSS-REFERENCE TO RELATED APPLICATIONS

This application claims the benefit of priority to U.S. Provisional Patent Application Nos. 61/231,782 and 61/231,784, which were filed on Aug. 6, 2009. Each of these patent applications is incorporated herein by reference in their entireties to the extent they do no conflict with the present disclosure.

### FIELD

This disclosure relates to medical devices and methods for removing a target molecule from a body fluid of subject, such as removal of amyloid beta from cerebrospinal fluid, and administering a therapeutic agent.

### BACKGROUND

A variety of disease states are thought to be associated with increased levels of a molecule in a subject. For example, amyloid beta or fibrils or plaques containing amyloid beta are associated with a variety of central nervous system diseases, such as Alzheimer's disease, Lewy body dementia and Down's syndrome. Recent therapeutic strategies designed to decrease plaque burden have focused on immunological approaches, including active and passive immunization targeting amyloid beta. One mechanism that is believed to be involved with these therapeutic strategies is removal of the soluble forms of amyloid beta (monomer and oligomer) from the CNS compartment, which in turn triggers the dissolution of unstable plaques due to a shift in the chemical equilibrium. However, such treatments involve administration of therapeutic substances into the patient and may be associated with risks of producing unintended immunologic and/or inflammatory conditions. To minimize such unintended effects, use of humanized antibodies has been proposed. However, production of such humanized antibodies tends to be costly.

One method for removal of target molecules from cerebrospinal fluid (CSF) of a subject is described in co-pending U.S. patent application Ser. No. 12/511,571, entitled APHERESIS OF A TARGET MOLECULE FROM CEREBROSPINAL FLUID, filed on Jul. 29, 2009, and having attorney docket number P0030601.01, which application is hereby incorporated herein by reference in its entirety to the extent that it does not conflict with the disclosure presented herein.

### BRIEF SUMMARY

The present disclosure describes devices, systems and methods that may be employed to accomplish removal of a target molecule, such as soluble amyloid beta components, from a body fluid, such as cerebrospinal fluid (CSF), of a subject and then returning the remaining components of the fluid to the fluid compartment of the subject. A therapeutic agent may additionally, or alternatively, be administered. In some embodiments, the therapeutic agent is delivered in the remaining components of CSF returned to the subject.

In an embodiment, a device is provided. The device includes an inlet, an outlet, and a medium having a solid support to which an antibody directed to a target molecule is bound. The device also includes a reservoir for containing the medium. The reservoir is operably coupled to the inlet and the outlet such that fluid may flow from the inlet, through the reservoir, to the outlet. The device further includes a second reservoir configured to house a therapeutic agent. The second reservoir is operably coupled to the outlet. The device additionally includes a pump operably coupled to the second reservoir and configured to cause fluid from the second reservoir to exit the outlet. The pump may also be operably coupled to the first reservoir and configured to cause fluid from the first reservoir to exit the outlet. Alternatively, the device may include a second pump operably coupled to the first reservoir and configured to cause fluid from the first reservoir to exit the outlet. Such devices may be used to remove a target molecule, such as amyloid beta, from CSF via apheresis by passing the CSF through the first reservoir containing the medium. A therapeutic agent housed in the second reservoir may be added to the CSF before it is returned to the subject. The device may be implantable.

In an embodiment, a method is provided. The method includes withdrawing fluid from a first cerebrospinal fluid (CSF) compartment of a subject and passing the withdrawn fluid through a reservoir of a medical device to remove the target molecule, such as amyloid beta, from the withdrawn fluid. The reservoir contains a medium having a solid support to which an antibody directed to a target molecule is bound. The media is capable of removing the target molecule from the CSF. The method further includes adding a therapeutic agent to the withdrawn fluid with removed target molecule, and delivering the withdrawn fluid with removed target molecule and added therapeutic agent to a second cerebrospinal fluid compartment of the subject. The first and second cerebrospinal fluid compartments may be the same or different.

Examples of therapeutic agents that may be administered by the devices described herein and in accordance with the methods described herein include ApoE-modulating agents; a RAGE inhibitors; β-secretase 1 (BACE1) inhibitors; γ-secretase inhibitors; muscarinic receptor subtype 1 (M1) agonists; growth factors; enzyme capable of degrading amyloid betas; mitochondrial antioxidants; insulin; and inhibitors of tumor necrosis factor (TNF). Such agents, or other therapeutic agents, may be added to the cerebrospinal fluid before the CSF exits the medical device and is returned to the subject.

In an embodiment, a method is provided. The method includes delivering directly to the central nervous system of the subject a therapeutic agent selected from the group consisting of an ApoE-modulating agent; a RAGE inhibitor; a β-secretase 1 (BACE1) inhibitor; a γ-secretase inhibitor; a muscarinic receptor subtype 1 (M1) agonists; a growth factor; an enzyme capable of degrading amyloid beta; a mitochondrial antioxidant; insulin; and an inhibitor of tumor necrosis factor (TNF). An anti-amyliod beta antibody may also be administered in addition to the therapeutic agents recited above. The method may be used to treat or study a disease associated with amyloid beta accumulation in a subject, such as Alzheimer's disease. The agents may be delivered directly to the CNS via any suitable route, such as directly to cerebrospinal fluid or intraparenchymally, e.g., to the hippocampus.

An example of an ApoE-modulating agent that may be administered is a liver X receptor (LXR) agonist. An example of a RAGE inhibitor that may be administered is PF-04494700. Examples of γ-secretase inhibitors that may be administered include r-flurbiprofen, MCP-7869, LY-450139, LY411575, and MK0752. Examples of M1 agonists that may be administered include cevimeline, talsaclidine, sabcomeline and milameline, xanomeline, and 5-(3-ethyl-1,2,4-oxadiazol-5-yl)-1,4,5,6-tetrahydropyrimmidine (CDD-0102). Examples of growth factors that may be administered include VEGF, BDNF, NGF, and IGF-1. Examples of enzymes capable of degrading amyloid beta are neprilysin, insulin-degrading enzyme (IDE), angiotensin-converting enzyme (ACE), and insulysin. An example of a microtubule stabilizing agent is the microtubule stabilizing compound peptide NAP (AL-108).

Various embodiments of the present invention provide several advantages over known methods and apparatuses for treating neurological disorders. By removing target molecules, such as soluble amyloid beta components, from cerebrospinal fluid (CSF) of a subject and then returning the CSF with removed target molecules back to the patient, delivery of exogenous therapeutic agents can be avoided. Adding therapeutic agents to the returned CSF can serve to augment the therapy or provide combinations of therapies that may not be feasible with administration of agents alone. By delivering the agents directly to a CSF compartment of a subject, peripheral side effects may be reduced and agents that may not be able to cross the blood-brain barrier in sufficient quantities may be used. These and other advantages will be evident to one of skill in the art upon reading the disclosure herein.

### BRIEF DESCRIPTION OF THE DRAWINGS

FIG. 1 is a schematic block diagram of a system interacting with a cerebrospinal fluid (CSF) compartment of a subject for removing amyloid beta from the CSF.

FIG. 2 is a schematic block diagram of a system interacting with a CSF compartment of a subject, showing details of an embodiment of media for removing amyloid beta from the CSF.

FIG. 3A is a schematic diagram of side view of a representative device and catheter with dashed lines revealing selected components within the device and catheter.

FIG. 3B is a schematic diagram of a longitudinal cross section of the catheter depicted in FIG. 3A.

FIG. 4 is a schematic diagram of a cross section of a brain and spinal cord showing CSF flow.

FIGS. 5-6 are diagrams of schematic views showing an implanted device and associated catheter in the environment of a subject.

FIG. 7 is a diagram of a schematic view showing an injection port in the environment of a patient.

FIGS. 8-15 are schematic block diagrams of selected components of representative systems.

FIGS. 16A-B are schematic diagrams of views of media having a solid support and a component capable of selectively binding amyloid beta.

FIGS. 17-24 are schematic block diagrams of selected components of systems including devices having a reservoir for housing an apheresis medium and a reservoir for housing a fluid composition containing a therapeutic agent.

FIG. 25 is a schematic diagram showing an implantable infusion system.

FIG. 26 is a schematic diagram of an implantable infusion system in a patient configured for intrathecal delivery.

FIGS. 27-28 are schematic diagrams of implantable infusion systems in patients configured for delivery to the patient's brain.

FIG. 29 is a schematic diagram of an implantable port for delivering agents to a patient's brain.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, the terms “treat”, “therapy”, and the like mean alleviating, slowing the progression, preventing, attenuating, or curing the treated disease.

As used herein, “disease”, “disorder”, “condition” and the like, as they relate to a subject's health, are used interchangeably and have meanings ascribed to each and all of such terms.

As used herein, “subject” means a mammal to which an agent, such as an antibody, is administered for the purposes of treatment or investigation. Mammals include mice, rats, cats, guinea pigs, hamsters, dogs, monkeys, chimpanzees, and humans.

As used herein, “coupleable” means capable of being coupled, directly or indirectly.

As used herein, “apheresis” means a process of removing a specific component from a fluid of a subject and returning the remaining components to the subject. Often apheresis includes returning the remaining components to same general fluid compartment from which the fluid was removed. For example, if blood is subjected to apheresis to remove a specific component, the remaining components may be returned to blood. If cerebrospinal fluid (CSF) is subjected to apheresis to remove a specific component, the remaining components may be returned to CSF.

Apheresis of a Target Molecule from Cerebrospinal Fluid

In various embodiments, the systems, devices and methods described herein may be used for treatment of any disease state for which apheresis of one or more molecules from a body fluid of a subject may be desirable or for investigation of the effects of removal of a molecule (as used herein, “removal” includes a reduction in concentration) from a body fluid of a subject and for which a therapeutic agent may be desirably administered. However for the purposes of brevity and clarity, much of the discussion presented herein is primarily directed to embodiments associated with the treatment or investigation of diseases for which removal of a target molecule from, or addition of a therapeutic agent to, cerebrospinal fluid may be desirable.

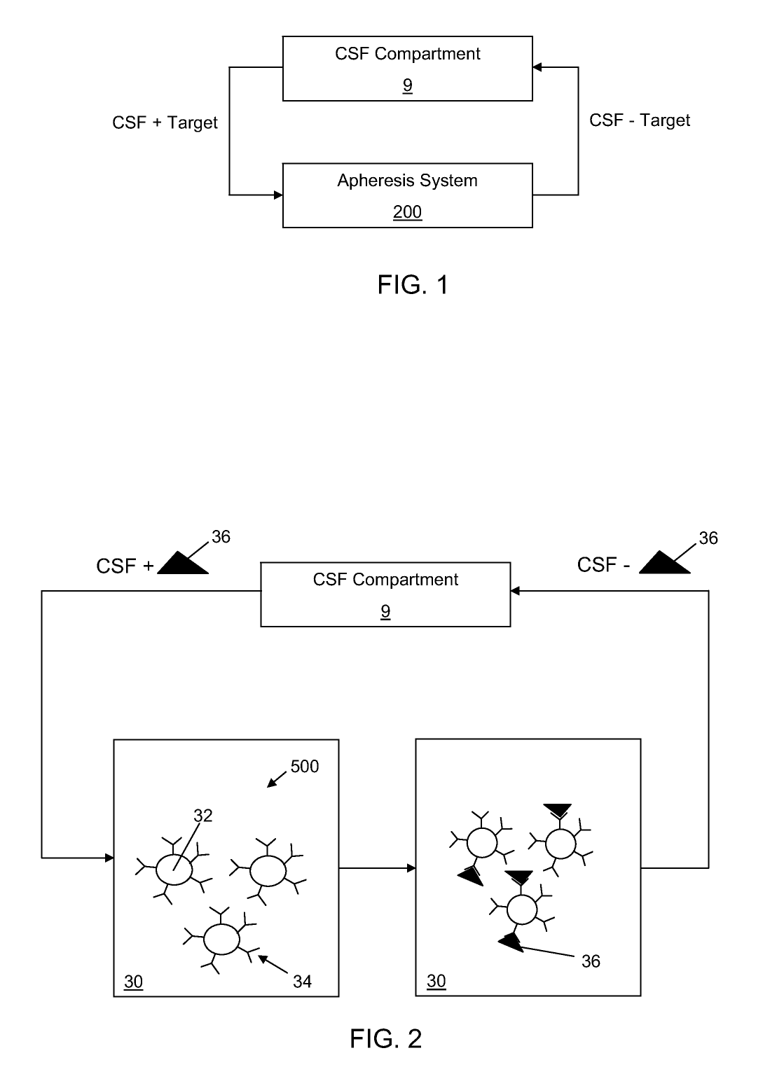

Referring to FIG. 1, an overview of an embodiment of a system is provided. Cerebrospinal fluid (CSF) is removed from a CSF compartment 9 of a subject, one or more target molecules, such as amyloid beta (Aβ), is removed from the CSF by an apheresis system 200 or device, and the CSF with removed target molecule is returned to a CSF compartment 9 of the subject. It will be understood that the CSF compartment 9 from which the CSF is removed may be the same or different compartment 9 to which the CSF is returned.

The apheresis system 200 or device may contain one or more components. In various embodiments, some or all of the components are implantable. In such embodiments, the implantable components having electrical parts are preferably contained within one or more hermetically sealed housings. In some embodiments, some or all of the components are external to the subject.

Referring now to FIG. 2, the apheresis system may include a reservoir 30 containing a medium 500 having a solid support 32 with which an antibody 34 or other binding partner that is capable of selectively binding target molecule 36 is associated. CSF containing the target molecule 36 is removed from a CSF compartment 9 of a subject, and the CSF is flowed through the reservoir 30 and contacted with the medium 500. The antibody 34 or other binding partner selectively binds the target molecule 36, removing some or all of the target molecule 36 from the CSF. The CSF with removed target molecule 36 is then returned to a CSF compartment 9, which may the same or different compartment 9 from which the CSF was removed.

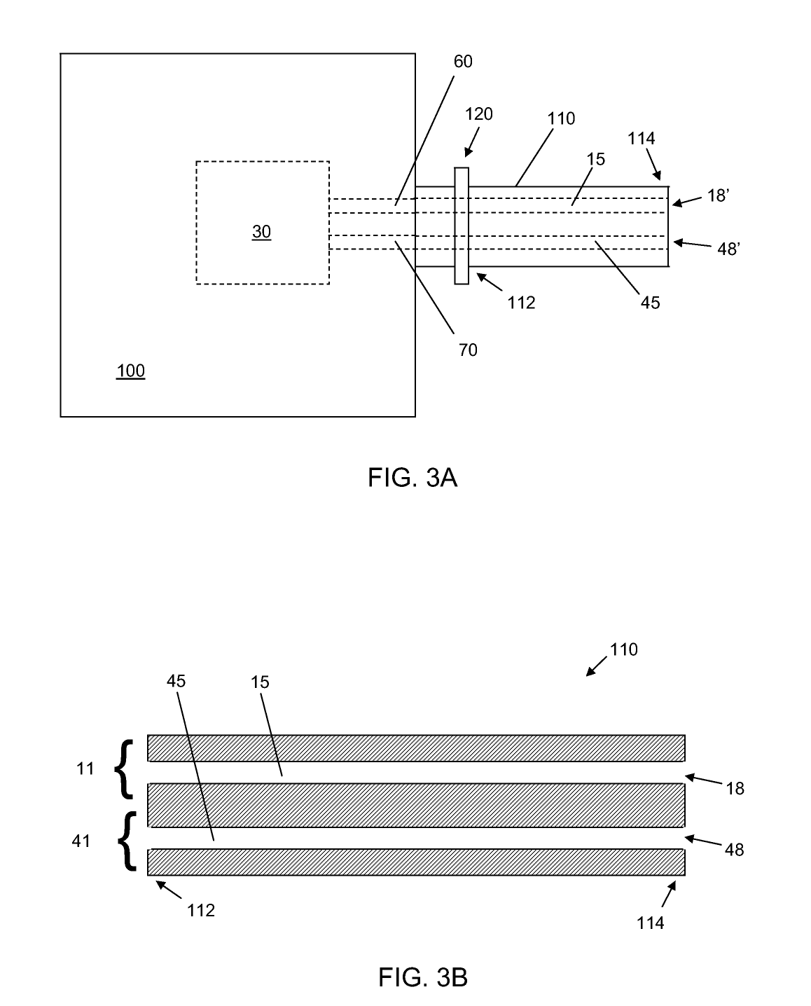

Referring now to FIG. 3A, a schematic diagram of a side view of a representative device 100 having a reservoir 30 operably coupled to a catheter 110 is shown (dashed lines indicate portions of device 100 or catheter 110 within and beneath the exterior surface the device or catheter). The device 100 may include a connector 120 to which proximal end 112 of catheter 110 may be connected. Catheter 110 may be connected to connector 120 via any suitable mechanism, such as clamping, compression fitting, interference fit, or the like. In the depicted embodiment, catheter 110 has a first lumen 15 and a second lumen 45. The first lumen 15 extends through the catheter 110 from proximal end portion 114 to an opening 18 at distal end portion 114. While not show, it will be understood that the opening 18 may be located at any position along the catheter 110 and there may be more than one opening. The first lumen 15 is operably coupled to an inlet 60 of device 100. While not shown, it will be understood that one or more components, such as a valve, a pump, or the like, may be located between the fluid flow path of inlet 60 and reservoir 30. The system is configured such that CSF may flow through opening 18, through first lumen 15, through inlet 60 and into reservoir 30.

Still referring to FIG. 3A, catheter 110, in the depicted embodiment, has a second lumen 45 that extends through the catheter 110 from the proximal end 112 to a delivery region 48 at distal end portion 114. While not shown, it will be understood that the delivery region 48 may be located at any position along the catheter 110 and there may be more than one delivery region. The second lumen 45 is operably coupled to an outlet 70 of device 100. While also not shown, it will be understood that one or more components, such as a valve, a pump, or the like, may be located between the fluid flow path of outlet 70 and reservoir 30. The system is configured such that CSF may flow from reservoir 30 through outlet 70, through second lumen 45 and out through delivery region 48.

CSF may enter reservoir 30, which contains a medium for removing a target molecule (see FIG. 2), via the first lumen 15 of catheter 110 and may exit reservoir 30 and be returned to a subject via delivery region 48 of catheter 110. While moving through reservoir 30, target molecules are removed from the CSF, and CSF with reduced levels of the target molecule is returned to the subject.

FIG. 3B depicts a schematic drawing of a longitudinal cross section of the catheter 110 shown in FIG. 3A. The depicted catheter 110 includes a first catheter 11 and a second catheter 41. In various embodiments, the first and second catheters 11, 41 are integrally formed in a single catheter 110. The first catheter 11 includes a lumen 15 extending through the catheter 11 from the proximal end portion 112 to the opening 18. The second catheter 41 includes a lumen 45 extending through the catheter 41 from the proximal end portion 112 to the delivery region 48. While a single catheter 110 with two lumens 15, 45 (or two catheters 11, 41) is depicted in FIG. 3, it will be understood that two separate catheters, or a catheter that splits into two separate catheters at some point along its length, may be employed.

### Cerebrospinal Fluid Compartment

In accordance with the teachings presented herein, CSF may be removed from or returned to any CSF compartment of a subject. One suitable CSF compartment for removal and return of CSF is the subarachnoid space. Cerebrospinal fluid is produced in the ventricular system of the brain and communicates freely with the subarachnoid space via the foramina of Magendie and Luschka.

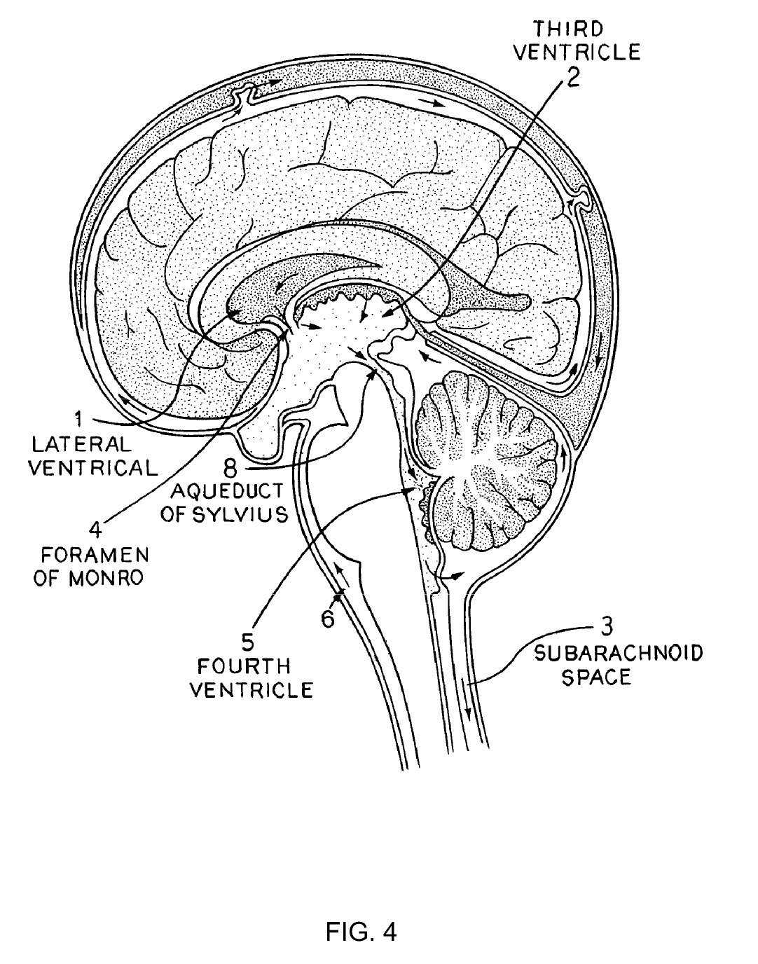

As illustrated in FIG. 4, the central nervous system (brain and spinal cord) is surrounded by cerebrospinal fluid 6 contained within the subarachnoid space 3. In addition, cerebrospinal fluid 6 is also contained in the four ventricles of the brain: two lateral ventricles 1, the third ventricle 2, and the fourth ventricle 5. The lateral ventricles 1 are connected to the third ventricle 2 via the foramen of Monro 4; the third ventricle 2 is connected to the fourth ventricle 5 via the aqueduct of Sylvius 8. The arrows within the subarachnoid space 3 in FIG. 4 indicate cerebrospinal fluid 6 flow.

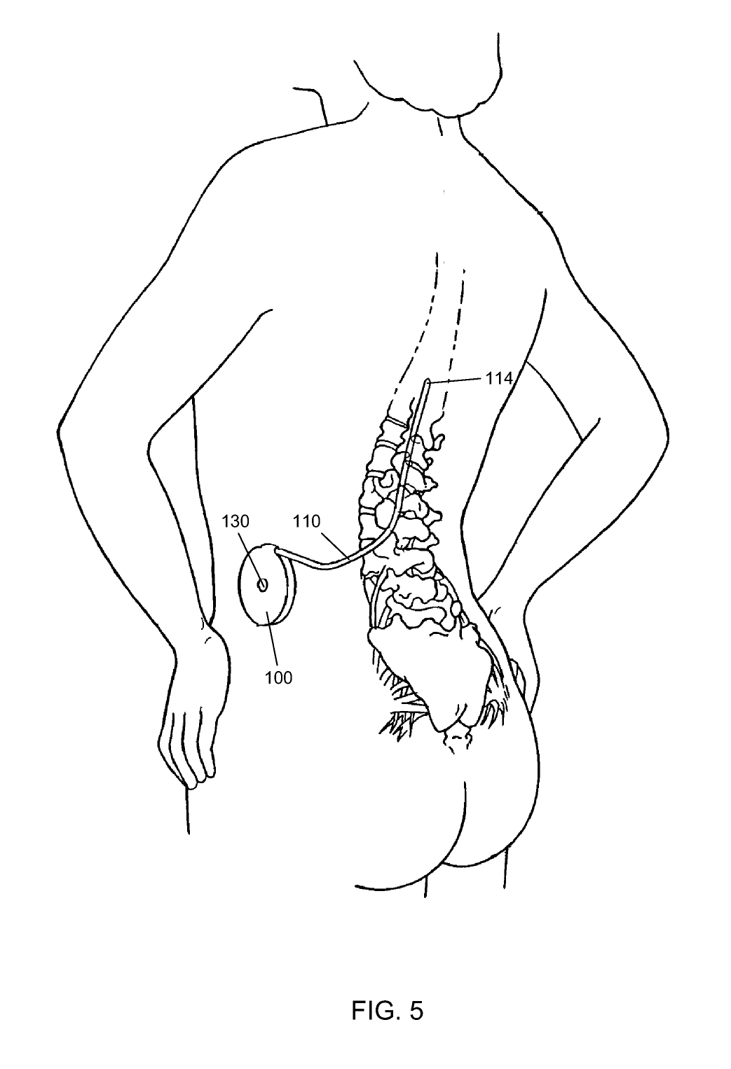

According to various embodiments, CSF is obtained from, or returned to, the spinal canal of a subject. With reference to FIG. 5, a system similar to that depicted in FIG. 3A is shown implanted in a patient. The system includes a device 100 containing a reservoir (not shown in FIG. 5) and catheter 110 operably coupled to the device 100. Distal portion 114 of catheter 110 is shown implanted in the intrathecal space of the patient's spinal canal. One or more openings (not shown in FIG. 5) for receiving CSF and one or more delivery regions (not shown in FIG. 5) for returning CSF are located at or near distal portion 114 of catheter 110. In the embodiment depicted in FIG. 5, device 100 is implanted below the skin of the patient. Preferably the device 100 is implanted in a location where the implantation interferes as little as practicable with activity of the patient. One suitable location for implanting the device 100 is subcutaneously in the lower abdomen. The device 100 may include a port 130 configured to fluidly communicate with reservoir (not shown in FIG. 5). A needle or other suitable device may be inserted into port 130 to inject or withdraw fluids from the reservoir, allowing for replacement or regeneration of the medium for removing a target molecule, as described in more detail below. Preferably, device 100 is implanted subcutaneously in a manner such that port 130 may be percutaneously accessed by a needle.

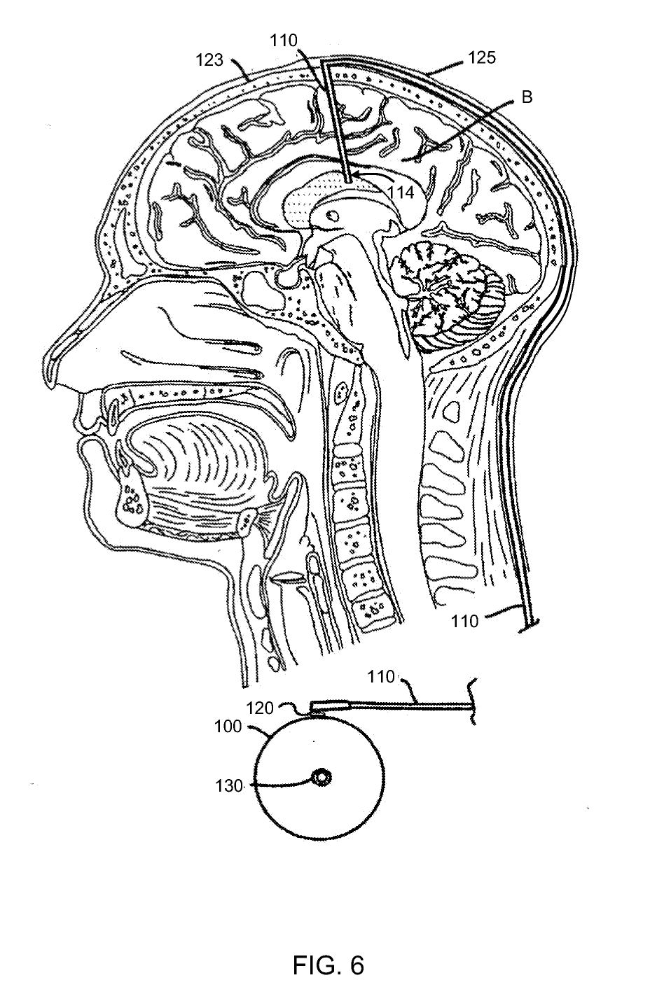

According to various embodiments, CSF may be withdrawn from, or returned to, a ventricle of the brain of a subject. Referring to FIG. 6, a device 100 having a reservoir (not shown in FIG. 6) containing media for removing a target molecule from the subject's CSF may be implanted below the skin of a subject. As with the device depicted in FIG. 5, the device 100 depicted in FIG. 6 may have a port 130 through which the reservoir may be accessed. In the depicted embodiment, the distal end 114 of catheter 110 terminates in a ventricle of the brain. Distal end portion 114 of catheter 110 may be implanted in the ventricle using conventional stereotactic surgical techniques. The distal portion 114 is surgically introduced through a hole in the skull 123 and a mid portion of catheter 110 may be implanted between the skull and the scalp 125 as shown in FIG. 6. Catheter 110 may be joined to implanted device 100, for example, via connector 120.

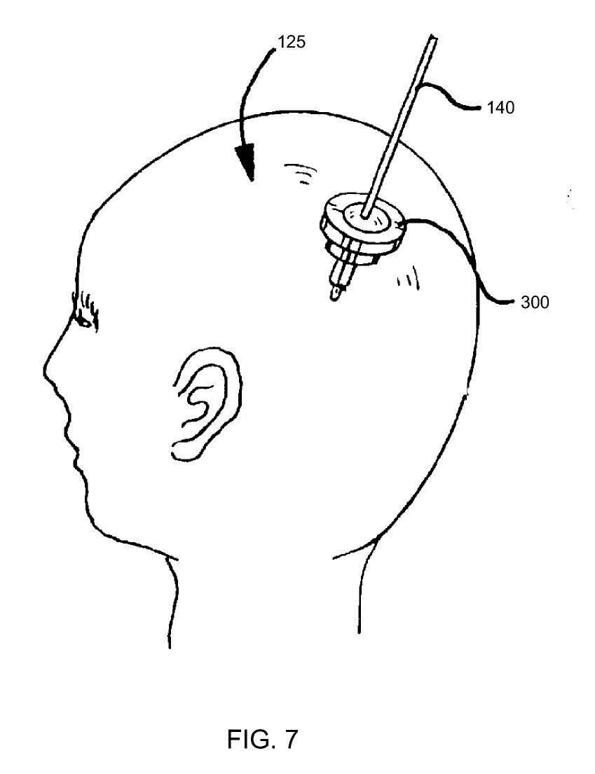

In various embodiments, CSF is removed from a subject, contacted with a medium for removing a target molecule from the CSF where the medium is contained in a reservoir external to the patient, and returned to the subject. The CSF may be removed from, or returned to, the subject via the subject's intrathecal space, intraventricular space, or the like. Referring to FIG. 7, CSF may be withdrawn from, or returned to, a subject's CNS via an injection port 300 implanted subcutaneously in the scalp of a patient 125, e.g. as described in U.S. Pat. No. 5,954,687 or otherwise known in the art. A guide catheter 140 may be used to guide a catheter for removing or returning CSF from the brain of the subject. Of course, a catheter for removing or returning CSF may be directly inserted through port 300 to the target location.

Any other known or developed implantable or external infusion device, port, shunt, or the like may readily be adapted for apheresis of amyloid beta from CSF.

### Representative Device Configurations

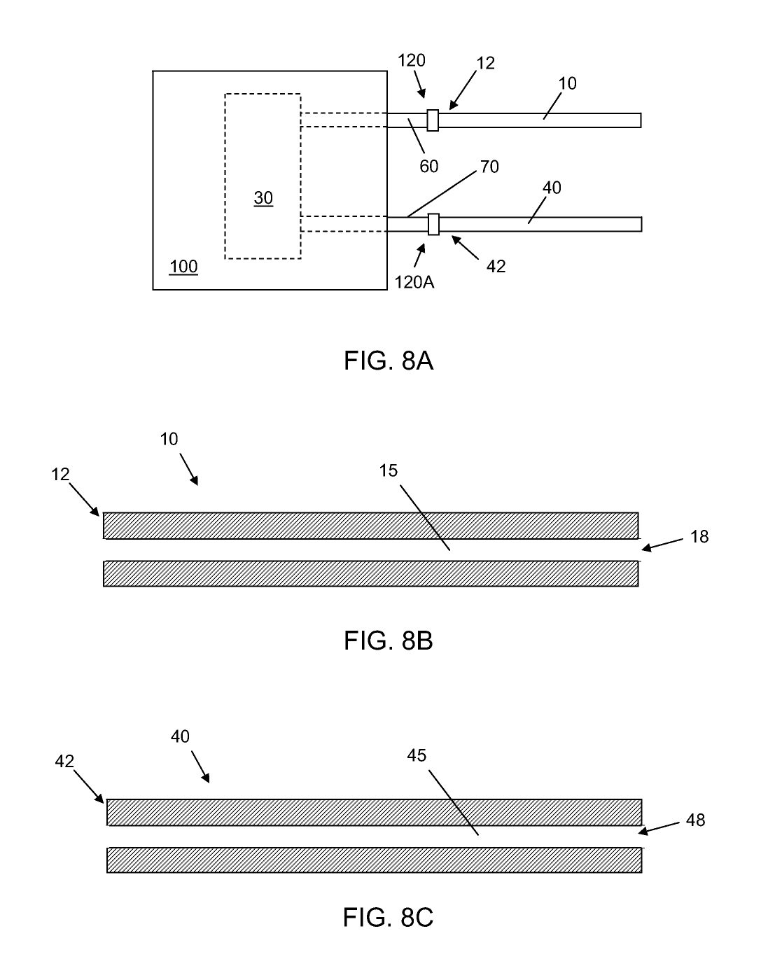

FIGS. 3A-B, 5 and 6, discussed above, and FIGS. 8-15 provide representative examples of device configurations that may be employed for apheresis of target molecule from CSF of a subject. It will be understood that components in addition to those depicted in FIGS. 3A-B, 5, 6 and 8-14 may be employed. However, selected components are shown for sake of clarity and brevity.

Referring to FIG. 8A, a device 100 having a reservoir 30 for containing a medium for removing amyloid beta from CSF is shown. The device 100 is similar to the device depicted in FIG. 3A, except that the device 100 in FIG. 8 is coupled to two catheters, a first catheter 10 or tube and a second catheter 40 or tube. The device 100 contains an inlet 60 and outlet 70 operably coupled to reservoir 30. A first catheter 10, a cross section of which is shown in FIG. 8B, is operably coupelable to the device 100, e.g. by securing via connector 120, such that lumen 15 of first catheter 10 can be in fluid communication with inlet 60. A second catheter 40, a cross section of which is shown in FIG. 8C, is operably coupleable to the device 100, e.g. by securing via connector 120A, such that lumen 45 of second catheter 40 can be in fluid communication with outlet 70. The system depicted in FIG. 8 is capable of operating such that, when opening 18 of first catheter 10 and delivery region 48 of second catheter 40 are placed in a CSF compartment of a subject, CSF may flow into opening 18, through lumen 15 of first catheter 10, through inlet 60, through reservoir 30, through outlet 70, through lumen 45 of second catheter 40, and out of delivery region 48. While CSF flows through reservoir 30 a medium contained within the reservoir can remove a target molecule from the CSF so that CSF with a reduced target molecule concentration may be returned to a CSF compartment of the subject.

While not shown in FIG. 8, it will be understood that the opening 18 may be located at any suitable position along the first catheter 10 and there may be more than one opening. In addition and while not shown, it will be understood that the delivery region 48 may be located at any suitable position along the second catheter 40 and there may be more than one delivery region. It will be further understood that one or more components, such as a valve, a pump, or the like, may be located between the fluid flow path of inlet 60 and reservoir 30 or outlet 70 and reservoir 30.

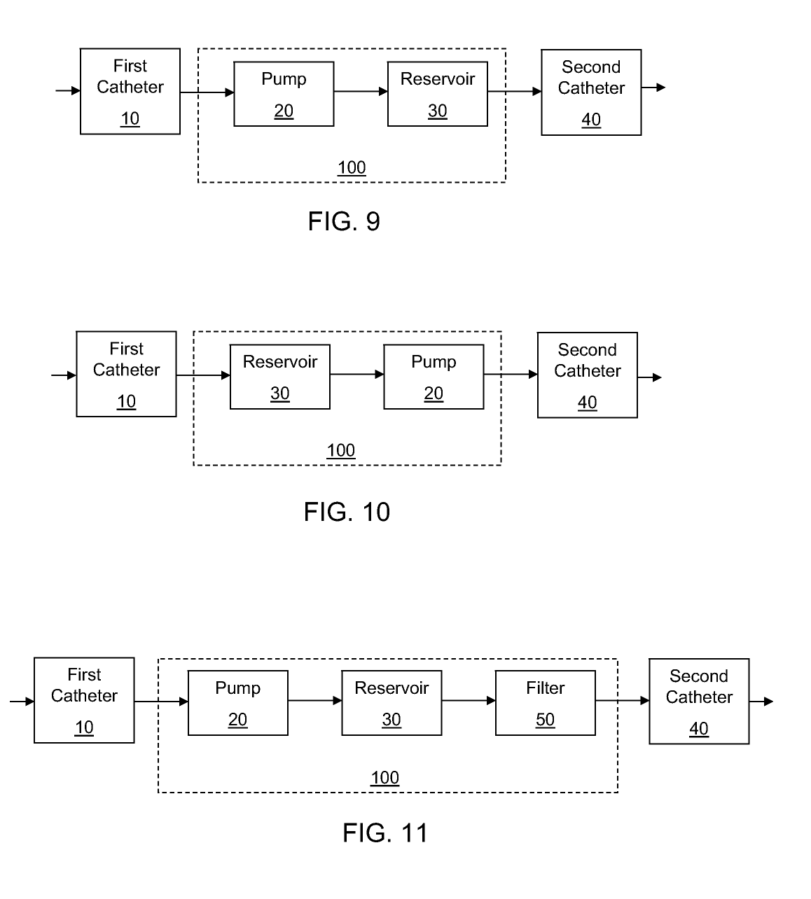

Referring now to FIGS. 9-12, block diagrams of representative systems are shown. The systems include a device 100 having a reservoir 30 and a pump 20 are shown. In some embodiments, apheresis systems do not include a pump. It is believed that the pulsatile nature of CSF flow may be sufficient to force CSF to flow through an apheresis system and back to CSF without use of a pump. In addition, or alternatively, gravity may be used to assist in pumpless devices; e.g. CSF may be removed from a higher CNS level and returned to a lower CNS level. While such pumpless systems and devices may be advantageous in some situations, it may be desirable, in some situations, to employ a pump; e.g., when media for removal of the target molecule restricts flow or to increase the amount of the target molecule removed by increasing the flow through the media. Any suitable pump 20 may be employed. For example, the pump 20 may be a peristaltic pump, an osmotic pump, a piston pump, a diaphragm pump, or the like. The pump 20 may be fixed rate, variable rate, programmable, etc.

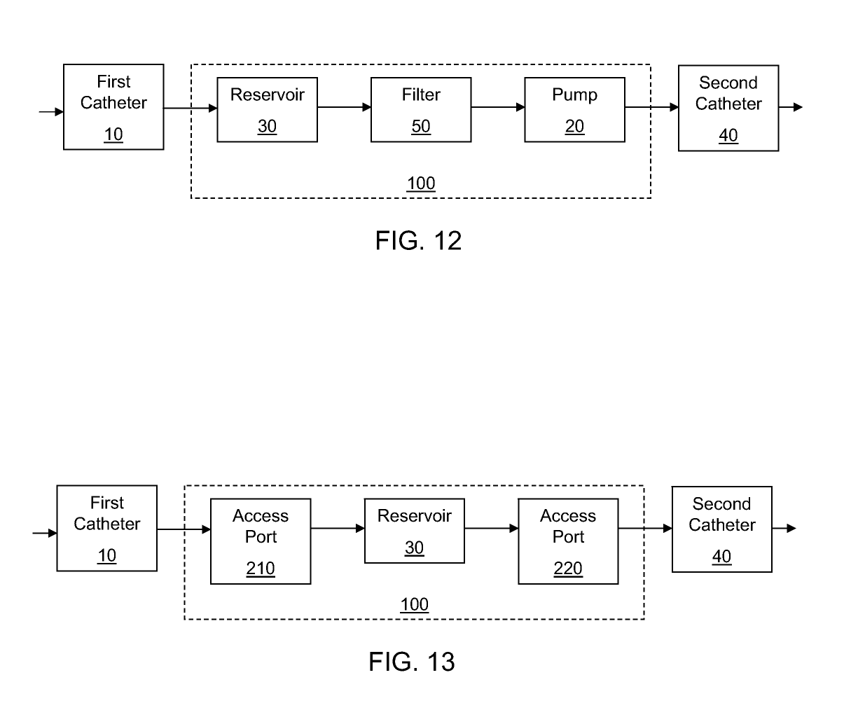

The systems depicted in FIGS. 9-12 further include a first catheter 10 and a second catheter 40, which may be a single catheter having two lumens (see, e.g., FIG. 3) or may be separate catheters (see, e.g., FIG. 8), operably coupled to the reservoir 30. The direction of the arrows in FIGS. 9-12 indicates the desired direction of flow of CSF through the system. As shown in FIG. 9 and FIG. 11, the pump 20 may be located upstream of the reservoir 30, i.e. between the reservoir 30 and the first catheter 40. Alternatively, as shown in FIGS. 10 and 12, the pump 20 may be located downstream of the reservoir 30, i.e. between the reservoir 30 and the second catheter.

The device may further include a filter 50. The filter 50 is configured to prevent selected components of media, such as a solid support bead, contained in the reservoir 30 from entering the subjects CSF via second catheter 40. In various embodiments, as described above, the media contains solid support material that may flow with CSF. In such embodiments, a filter 50 may be desirable. In some embodiments, the media contains a solid support that is not likely to flow or will not flow with CSF as the CSF passes through the reservoir 30. In such embodiments, it may be desirable to omit a filter 50 from the device. Preferably, the filter 50 is positioned such that it prevents selected components of the media from leaving the reservoir 30. In various embodiments, filter 50 is positioned within the reservoir 30 or immediately downstream of the reservoir 30. Filter 50 may be made of any suitable material, such as poly(tetrafluoroethane) (PTFE), nylon, cellulose, mixed cellulose ester, or polyvinylidene difluoride (PVDF). Preferably, the pore size of filter 50 is small enough to retain the solid support, such as beads. For example, the pore size may be about 20 to about 50 microns less in diametric dimension than the diametric dimension of the solid support. In various embodiments, it may be desirable for the filter to exhibit a low affinity for binding to protein. One suitable low protein binding material is PVDF. In some embodiments, a filter 50 may serve as a solid support for an antibody for binding amyloid beta.

Referring now to FIG. 13, a block diagram of a system having access ports 210, 220 upstream and downstream of the reservoir 30 is shown. Access ports 210 may be used to sample CSF before it enters the reservoir; e.g. to assess the level of target molecule in the CSF prior to apheresis. Access port 220 may be used to sample CSF after apheresis; e.g. to determine how effectively the target molecule is being removed from the CSF by the device 100. CSF removed via access portion 210, 220 may be used to determine the effects of apheresis on molecules other than the target molecules, to monitor or diagnose a condition of the subject, to determine when the media for removal of the target molecule is saturated, or the like. For example, the concentration of a target molecule or other molecule in fluid sampled from an access port 210, 220 may be quantitatively or semi-quantitatively determined via a suitable assay or device, such as an ELISA assay or microchip based bioassay. The amount of target or other molecule may be used to determine whether apheresis is effectively removing the target molecule or having a desirable or expected effect on another molecule, may be used to determine whether the apheresis media is, or is becoming, saturated, or the like. Apheresis parameters may be altered based on information regarding concentration of the target or other molecule. For example, if an apheresis device includes a variable rate or programmable pump, the rate at which fluid is flows through the apheresis media may be changed to remove more (increase flow) or less (decrease flow) of the target molecule from the CSF of the patient.

In some embodiments (not shown in FIG. 13), a system includes only one access port for sampling CSF. The single access port may be upstream, downstream, or at the reservoir.

While not shown in FIGS. 9-13, it will be understood that the devices 100 may include further components such as an inlet, an outlet, a microprocessor for controlling the pump, a sensor module, a telemetry module, a diagnostics module, a power supply, a reservoir access port, etc.

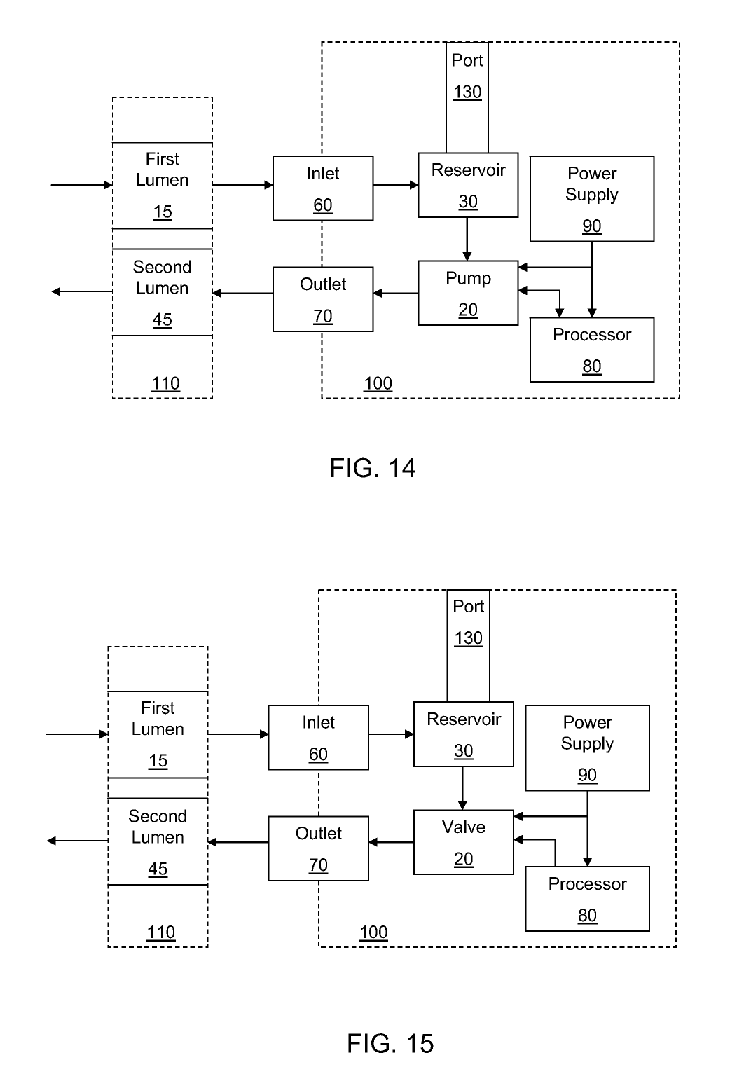

Referring now to FIG. 14, a block diagram of a system is shown. The system includes a catheter 110 through which CSF may flow and which is configured to be implanted into the CNS of a subject, and a device 100 for removing a target molecule from the CSF. The device 100 includes a reservoir 30 and a pump 20 operably coupled to the reservoir 30 and configured to cause CSF to move through the reservoir 30. The device 100 further includes a processor 80 operably coupled to the pump 20 and configured to control the rate at which the pump 20 causes CSF to move through the reservoir 30. The device also includes a power supply 90 operably coupled to the processor 80. The power supply may also be operably coupled to the pump 20 in embodiments where it is desirable to provide power for one or more aspects of pump operation.

With reference to FIG. 15, a block diagram of an alternative embodiment of a system is shown. The system includes first 10 and second 40 catheters through which CSF may flow operably coupled to a device 100 for removing a target molecule from the CSF. The device 100 includes a valve 220 configured to control the rate of flow of CSF through the device 100. The valve 220 is operably coupled to reservoir 30. While only one valve 220 is depicted, it will be understood that device 100 may include more than one valve. Power supply 90 and processor 80 are operably coupled to valve 220. Processor 80 may be configured to control the rate at which CSF may flow through the valve 220, e.g. by causing the valve 220 to open or close, partially or entirely, as instructed. While not shown, it will be understood that device 100 may include a pump or a sensor for detecting flow. The sensor may be coupled to the processor 80 to allow for closed loop control of valve 20.

The devices 100 shown in FIGS. 14-15 also include a port 130 for accessing the reservoir 30. A needle (not shown) may be introduced into port 130 to introduce or remove fluid from the reservoir 30.

The device and system configurations described herein are representative examples of configurations that may be employed. It will be understood that the various system components shown in FIGS. 3A-B, 5, 6, and 8-15 may be readily interchanged as desired. For example, an access port 210, 220 depicted in FIG. 13 may be readily introduced into a device or system configuration depicted in FIGS. 3A-B, 5, 6, 8-12, 14 or 15. It will be also be understood that other configurations and other components are readily obtainable and are contemplated for use with the teachings described herein.

### Target Molecule

The apheresis systems described herein may be used to treat any disease in which removal of a target molecule from a bodily fluid may be beneficial or to investigate the effects of removal of a target molecule from a body fluid of a subject (e.g., in experimental animals). For the purposes of brevity, much of this disclosure is limited to a discussion regarding removal of a target molecule from CSF and diseases for which such removal may be beneficial, such as Alzheimer's Disease (AD), Lewy body dementia, and Down's Syndrome. Any one or more target molecules may be removed from CSF via apheresis as described herein. For the purposes of brevity a few examples of target molecules that may be removed from CSF are discussed below.

In various embodiments, the target molecule to be removed from CSF via apheresis is tau. Tau is a microtubule-associated protein that is found mostly in neurons. One function of tau is to modulate the stability of axonal microtubules. However, hyperphosphorylation or excessive tau activity may result in self-assembly of tangles of paired helical filaments or straight filaments, thought to be involved in AD and other diseases. Accordingly, apheresis of tau may result in a reduction of self-assembly of tangles. To date there are six known isoforms of tau. Any one or more of the six isoforms of tau may be a target molecule for apheresis as described herein. Phosphorylated or unphosphorylated tau may be removed via apheresis.

In various embodiments, one or more cytokines, such as interleukin (IL)-11, IL-18, or tumor necrosis factor-alpha (TNFα), may be a target molecule for CSF apheresis, as intrathecal inflammation has been reported to precede development of AD. Some anecdotal reports and a pilot study have shown that anti-TNFα therapies may be beneficial for AD patients. However, as anti-TNFα therapeutic agents are biologics, their cost can be prohibitive. Apheresis may be prove to be a less expensive alternative, where media containing a TNFα antibody or binding partner can be used to remove a significant amount of TNFα with a relatively small amount of antibody or other binding partner.

In some embodiments, soluble TNF receptors are target molecules for apheresis of CSF, as soluble TNF receptors may be associated with Aβ metabolism and conversion to dementia in subjects with mild cognitive impairment. Any suitable TNF receptor, such as CD120a, CD120b or other TNF receptors may be a target molecule.

In various embodiments, one or more α- or γ-synuclein proteins are target molecules for CSF apheresis. Alpha- and γ-synuclein proteins have been found to be present in CSF and are increased in aged subjects with neurodegenerative and vascular changes. Alpha-synuclein is a structural component of Lewy body fibrils. Three point mutations have been identified in α-synuclein in some familial forms of Parkinson's disease (A53T, A30P, and E46K). In some embodiments, one or more of these mutated forms of α-synuclein may be target molecules for apheresis.

In some embodiments, Apolipoprotein E is a target molecule for apheresis of CSF. Apolipoprotein E may be a desirable target in any patient suffering from or at risk of AD or other dementia, and may be a particularly desirable target in patients carrying at least one allele of ApoE-ε4 or ApoE-ε3.

In some embodiments, BACE1 (also called β-secretase or memapsin-2) may be a target molecule for apheresis of CSF. BACE1, which cleaves at the β-site of amyloid precursor protein (APP), is thought to be involved in the pathogenesis of AD and other dementias. When APP is cleaved by BACE1 and γ-secretase results in the production of amyloid beta (Aβ), which is also a target molecule for CSF apheresis in some embodiments.

As used herein, “beta amyloid”, “amyloid beta”, “Abeta” and “Aβ” are used interchangeably. Aβ is peptide of about 39-43 amino acids that corresponds to a peptide formed in vivo upon cleavage of an amyloid beta A4 precursor protein (APP or ABPP) by beta-secretase (at the N-terminal portion of Aβ) and gamma secretase (at the C-terminal portion of Aβ). See, e.g., Strooper and Annaert (2000; J. Cell Sci., 113, 1857-1870) and Evin and Weidemann (2002; Peptides, 23, 1285-1297). The most common isoforms of Aβ are Aβ40 and Aβ42, 40 and 42 amino acids, respectively. Aβ42 is less common, but is thought to be more fibrillogenic than Aβ40. Effective antibodies or binding partners may bind both Aβ40 and Aβ42, selectively bind Aβ42, bind all or some isoforms of Aβ, or the like.

Aβ is the main constituent of amyloid plaques in brains of Alzheimer's disease patients. Similar plaques can also be found in some Lewy body dementia patients and Down's Syndrome patients. Similar plaques or Aβ aggregates are found in the cerebral vasculature of cerebral amyloid angiopathy patients. More recent reports describe the accumulation of both soluble and intracellular Aβ ahead of the extracellular amyloid plaques forming (in all of the conditions above) in earlier disease states. In various embodiments, the systems, devices, or methods described herein may be employed to treat or prevent such diseases.

It will be understood that clearance of soluble forms of Aβ or fibrils or plaques containing Aβ are contemplated. Current models of the physical state of Aβ are evolving. Over about the last 20 years, researchers have defined the soluble toxic species of Aβ according to multiple synonyms. The antibodies described herein may target any of the species defined in Masters and Beyreuther's review (2006), Brain, Nov; 129(Pt 11):2823-39. Targets include soluble dimmers, tetramers, dodecomers that may ultimately form oligomers, oligomers, amorphous aggregates, Abeta derived diffusible ligands (ADDLS), β-balls, β-Amy balls, globular Aβ oligomer, paranuclei, preamyloid, protofibril, spherocylindrical miscelles, spherical particles, spherical prefibrillar aggregates, and toxic Aβ soluble species.

In some embodiments, a target molecule for apheresis of CSF is one or more of β-secretase (BACE)-cleaved soluble amyloid precursor proteins (sAPPβ), N-terminal fragments of APP, truncated APP or Aβ polypeptides, C-terminal truncated Aβ polypeptides, and the like.

In some embodiments, one or more of prostaglandin-d-synthase-transthyretin protein complex, isoprostane, toxic advanced glycation end-products (TAGE), and light chain, heavy chain or hyperphosphorylated heavy chain neurofilaments may be target molecules for apheresis of CSF. Each of these molecules may be associated with AD or other forms of dementia.

### Binding Partner

Any suitable binding partner may be employed to remove a target molecule from CSF of a subject. A “binding partner” means any molecule which has selective binding affinity for the target molecule. Binding partners can include, without limitation, proteins, peptides, nucleic acids, amino acids, nucleosides, antibodies, antibody fragments, antibody ligands, aptamers, peptide nucleic acids, small organic molecules, lipids, hormones, drugs, enzymes, enzyme substrates, enzyme inhibitors, coenzymes, inorganic molecules, polysaccharides, and monosaccharides. As used herein, the term “selective binding affinity” means greater affinity for non-covalent physical association or binding to selected molecules relative to other molecules in a sample under appropriate conditions. Examples of selective binding affinity include the binding of polynucleotides to complementary or substantially complementary polynucleotides, antibodies to their cognate epitopes, and receptors to their cognate ligands under appropriate conditions (e.g., pH, temperature, solvent, ionic strength, electric field strength). Selective binding affinity is a relative term dependent upon the conditions under which binding is tested, but is intended to include at least a 2× greater affinity for amyloid beta than any non-target molecules present in a sample under appropriate conditions. If a test sample includes more than one type of target molecule (e.g., allelic variants from one locus), a binding partner can have selective binding affinity for one or more of the different target molecules relative to non-target molecules.

In many embodiments, a binding partner is an antibody, which can be readily produced or can be purchased from a commercial vendor such as Covance, Inc., Millipore, or AbD Serotec. Any antibody directed to a target molecule may be employed in accordance with the teachings presented herein. Exemplary antibodies include polyclonal, monoclonal, and humanized antibodies.

The term “antibody” is used in the broadest sense and specifically includes, for example, single monoclonal antibodies, antibody compositions with polyepitopic specificity, single chain antibodies, and fragments of antibodies (see below). The term “monoclonal antibody” as used herein refers to an antibody obtained from a population of substantially homogeneous antibodies, i.e., the individual antibodies forming the population are identical except for possible naturally-occurring mutations that may be present in minor amounts. An antibody may include an immunoglobulin constant domain from any immunoglobulin, such as IgG-1, IgG-2, IgG-3, or IgG-4 subtypes, IgA (including IgA-1 and IgA-2), IgE, IgD or IgM. In various embodiments, an antibody includes a combination of various immunoglobulin isotypes, either to a specific epitope of anti-amyloid or broader spectrum IgGs.

“Single-chain Fv” or “sFv” antibody fragments include the V_{H} and V_{L} domains of antibody, wherein these domains are present in a single polypeptide chain. Preferably, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The antibody may be directed towards any region of a target molecule. For example, for Aβ, antibodies may be directed to an epitope at the N-terminal region of Aβ, e.g., the epitope contains amino acids within 5 amino acids of the N-terminal amino acid. In some embodiments, the epitope lies within amino acids 3-8 of an Aβ peptide and corresponds to amino acids 1-17. In some embodiments, antibodies are directed at the mid-terminal region of Aβ, e.g., the epitope corresponds to amino acids 17-24 of human Aβ. In various embodiments, antibodies are directed to an epitope at the C-terminal region of Aβ, e.g., the epitope corresponds to amino acids 24-40/42/43 of human Aβ or contains amino acids within 5 amino acids of the C-terminal amino acid.

Any known or developed method for preparing antibodies may be used.

### A. Polyclonal Antibodies

Polyclonal antibodies can be raised in a mammal, for example, by one or more injections of an immunizing agent and, if desired, an adjuvant. Typically, the immunizing agent or adjuvant will be injected in the mammal by multiple subcutaneous or intraperitoneal injections. The immunizing agent may include the target molecule or fragment or fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

### B. Monoclonal Antibodies

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the target molecule or fragment or fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell, e.g. as described in Goding, Monoclonal Antibodies: Principles and Practice, Academic Press, (1986) pp. 59-103 Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell murine myeloma lines can be obtained, for example, from the Salk Institute Cell Distribution Center, San Diego, Calif. and the American Type Culture Collection, Manassas, Va. Human myeloma and mouse-human heteromyeloma cell lines also have been described for the production of human monoclonal antibodies See, e.g., Kozbor, J. Immunol., 133:3001 (1984); and Brodeur et al., Monoclonal Antibody Production Techniques and Applications, Marcel Dekker, Inc., New York, (1987) pp. 51-63.

The culture medium in which the hybridoma cells are cultured can then be assayed for the presence of monoclonal antibodies directed against the target molecule. For example, the binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods; e.g., as described in Goding, Monoclonal Antibodies: Principles and Practice, Academic Press, (1986) pp. 59-103. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (see, e.g., U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody, or can be substituted for the variable domains of one antigen-combining site of an antibody to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. For example, antibodies may be digested with papain digestion to form F(ab)′₂ fragments.

### C. Human and Humanized Antibodies

Humanized forms of non-human (e.g., murine) antibodies may be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin or that eliminate or reduce T-cell epitopes from the non-human antibodies. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also include residues which are found neither in the recipient antibody nor in the imported CDR or framework sequences. In general, the humanized antibody will include substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also include at least a portion of an immunoglobulin constant region (Fc), typically that of a human immunoglobulin. See, e.g., Jones et al., Nature, 321:522-525 (1986); Riechmann et al., Nature, 332:323-329 (1988); and Presta, Curr. Op. Struct. Biol., 2:593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (see, e.g., Jones et al., Nature, 321:522-525 (1986); Riechmann et al., Nature, 332:323-327 (1988); and Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries. See, e.g., Hoogenboom and Winter, J. Mol. Biol., 227:381 (1991); and Marks et al., J. Mol. Biol., 222:581 (1991). Of course other techniques, such as those described by Cole et al. and Boerner et al., are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection or mutagenesis methods. Affinity matured antibodies may have an affinity that is five time or more than the starting antibody (generally murine, humanized or human) from which the matured antibody is prepared.

Other methods for humanizing antibodies that may be employed include those described in, e.g., EP0629240, EP0983303, and PCT/GB06/000355, where methods for reducing or eliminating T cell epitopes are discussed.

In numerous embodiments, a humanized anti-Aβ antibody as described in U.S. Provisional Patent Application Ser. No. 60/990,401, entitled “Humanized Anti-Amyloid Beta Antibodies”, filed Nov. 27, 2007, and having attorney docket no. 30103.00 is employed according to the teachings presented herein.

### Media

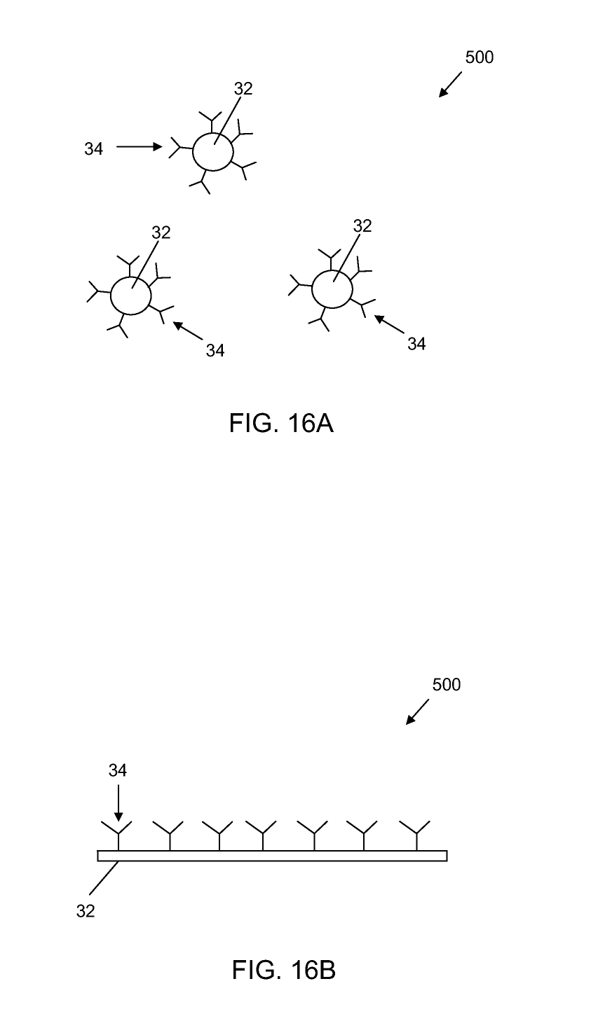

Referring now to FIGS. 16A-B, a medium 500 for removing a target molecule from CSF as described herein may include a solid support 32 and an antibody 34 or other suitable binding partner capable of selectively binding the target molecule. The solid support 32 may be porous or non-porous and may be of any convenient shape. For example, the solid support 32 may be in the form of beads (FIG. 16A), a membrane (FIG. 16B), hollow fiber, or the like. The solid support 32 may be made of any suitable material, such as glass, plastic polymer, polysaccharides, nylon, nitrocellulose, or TEFLON. Examples of suitable materials for beads include agarose, sepharose, dextran, polymethacrylate, polyacrylamide, silica and cellulose. In some embodiments the beads are paramagnetic, such as DynaBeads available from Baxter Immunotherapy Group, Santa Ana, Calif. Examples of suitable materials for membranes or fibers include cellulose, polysulfone, and polyamide. If the solid support 32 material is porous, the pores may be of any suitable size. In various embodiments, the pores are of a diameter of between about 300 to about 700 angstroms.

As shown in FIG. 16A-B, one or more antibody 34 or other suitable binding partner directed to the target molecule may be bound to a solid support 32 such as a bead or membrane. The antibodies may be bound to any surface of the support 32. For example, and while not shown in FIG. 16B, the antibodies 34 or binding partner may be bound to first and second opposing major surfaces of a membrane 32.

Antibodies or other suitable binding partners may be bound to the solid support via any suitable mechanism. As used herein, “binds”, “bind”, “bound”, “binding” or the like, in the context of an antibody 34 to a solid surface 32, refers to an association of the antibody 34 with the solid surface 32 that retains the antibody 34 in close proximity to the solid surface 32 when CSF flows through a reservoir containing media including the solid surface 32 with bound antibody 34.

The “binding” may be covalent or non-covalent. Examples of non-covalent binding include non-specific adsorption, binding based on electrostatic (e.g. ion, ion-pair interactions), hydrophobic interactions, hydrogen bonding interactions, surface hydration force and the like. Any suitable technique for non-covalently binding an antibody 34 or other suitable binding partner to a solid support 32 may be employed. For example, antibodies 34 may be attached to a solid support 32 by using protein A or G (bacterial cell wall proteins) which have high affinity to the constant (Fc) regions of antibodies. These proteins interface between the solid support 32 and the antibody 34. Protein A or G may be covalently attached to the solid support by using reductive amination, a cyanogen bromide technique, a gluteraldehyde method, or another suitable technique. Similarly, any suitable technique for covalently binding an antibody 34 or other suitable binding partner to a solid support 32 may be employed. Covalent immobilization of an antibody 34 or other suitable binding partners to a solid support 32 often involves activation of the antibody 34 or other suitable binding partners or the support 32. One example is the creation of aldehydes in the carbohydrate regions of an antibody 34 for its attachment to a support 32 that contains amines or hydrazide groups. Activation of the support 32 includes immobilization of antibodies 34 through their amine groups to supports 32 activated with N-hydroxysuccinimide or carbonyldiimidazole. Other methods used to link amine-containing antibodies 32 or other suitable binding partners to solid supports 34 include the cyanogen bromide method and reductive amination. Antibodies 34 or other suitable binding partners may also be attached to supports 32 using sulfhydryl-reactive methods which include haloacetyl, maliemide, and pyridyl disulfide methods. Antibodies 32 or other suitable binding partners may also be covalently linked to solid supports 34 using hydroxyl-reactive, carbonyl-reactive, or carboxyl-reactive methods.

In various embodiments, an Fc portion of an antibody 34 is bound to the solid support 32.

It will be understood that some antibody 34 may be eluted from the solid support 32 and may enter a CSF compartment of the subject with return of the CSF during apheresis. In such circumstances, it may be desirable to employ a humanized antibody when performing apheresis in a human to reduce the chance of developing an adverse immune reaction to the eluted antibody. In circumstances where little or no antibody 34 elutes from the solid support 32, it may be desirable to employ non-humanized antibodies as such antibodies are more readily obtainable in large quantities.

To maximize the capacity of the media 500 to remove the target molecule from CSF, the density of the antibody 34 or other suitable binding partner bound to the support 32 may be maximized. The density of the antibody 34 or other suitable binding partner bound to the support 32 can be readily controlled by varying the concentration of antibody 34 used to bind to the support 32.

When the media 500 containing the antibodies 34 or other suitable binding partners bound to the solid support 32 becomes saturated, fully or partially, with the target molecule removed from CSF, the media 500 may be replaced or regenerated. If the solid support 32 with bound antibody 34 or other suitable binding partners can flow through a syringe, such as with many beads, the media may readily be replaced. For example, a syringe needle or other suitable catheter may be inserted into a port 130 (see, e.g., FIGS. 5-6 and 14-15) to withdraw the medium containing the solid support 32 with bound antibody 34 or other suitable binding partners from a reservoir 30 and fresh medium may be introduced into the reservoir 30.

In various embodiments, solid surface is regenerated. As used herein, “regenerated”, in the context of media containing a solid support 32 with a bound antibody 34 or other suitable binding partners capable of binding a target molecule, means that the ability of the media to remove the target molecule is improved. Regeneration may include eluting the target molecule from the solid support with bound antibody. For example, an elution buffer may be added to the reservoir containing the media and later removed with eluted target molecule. Examples of solutions that may be used to elute target molecule from the antibody 32 include (i) low pH solutions (e.g., pH of about 1 to about 2.5) using, for example phosphate, citric, formic, or acetic acid, (ii) solutions having chaotropic agents, such as potassium or sodium thiocyanate at concentrations of about 1.5 M to about 3 M, sodium iodide at concentrations of about 2.5 M to about 3.0 M, or sodium chloride at concentrations of about 2M to about 4 M, and (iii) the like. It may be desirable to rinse the reservoir and media prior to resuming CSF flow through the reservoir. The reservoir may be rinsed with any physiologically acceptable solution, such as water, phosphate buffered saline, and the like.

The amount of target molecule removed from the media during regeneration of the media can be quantitatively or semi-quantitatively determined. Such information can be used to determine whether apheresis is effectively removing the target molecule, determine whether parameters should be altered (e.g., increase or decrease fluid flow through media, alter the concentration of the target molecule binding partner in the media, alter the specificity of the binding partner, etc.), or the like.

### Apheresis Methods of Treatment or Study

Apheresis of a target molecule from CSF of a subject may be employed to treat or study a variety of disease states. In various embodiments, apheresis as described herein is used to treat or study a disease associated with increased or aberrant soluble Aβ, amyloid fibrils or amyloid plaques. Examples of disease associated with increased or aberrant soluble Aβ, amyloid fibrils or amyloid plaques include Alzheimer's disease (AD), cerebral amyloid angiopathy (CAA), Lewy body dementia, and Down's syndrome (DS).

In various embodiments a method includes identifying a subject suffering from or at risk of AD and removing Aβ or another target molecule from the patient's CSF via apheresis. Those at risk of AD include those of advancing age, family history of the disease, mutations in APP or related genes, having heart disease risk factors, having stress or high levels of anxiety. Identification of those suffering from or at risk of AD can be readily accomplished by a physician. Diagnosis may be based on mental, psychiatric and neuropsychological assessments, blood tests, brain imaging (PET, MRI, CT scan), urine tests, tests on the cerebrospinal fluid obtained through lumbar puncture, or the like.

In various embodiments a method includes identifying a subject suffering from or at risk of CAA and removing Aβ or another target molecule from the patient's CSF via apheresis. Symptoms of CAA include weakness or paralysis of the limbs, difficulty speaking, loss of sensation or balance, or even coma. If blood leaks out to the sensitive tissue around the brain, it can cause a sudden and severe headache. Other symptoms sometimes caused by irritation of the surrounding brain are seizures (convulsions) or short spells of temporary neurologic symptoms such as tingling or weakness in the limbs or face. CAA patients can be identified by, e.g., examination of an evacuated hematoma or brain biopsy specimen, the frequency of APOE ε2 or ε4 alleles, with clinical or radiographic (MRI and CT scans) grounds according the Boston Criteria (Knudsen et al., 2001, Neurology; 56:537-539), or the like. Those at risk of CAA include those of advancing age, those having the APOE genotype, and those having other risk factors associated with AD.

In some embodiments a method includes identifying a subject suffering from or at risk of Down's syndrome and removing Aβ or another target molecule from the patient's CSF via apheresis. A newborn with Down's syndrome can be identified at birth by a physician's physical exam. The diagnosis may be confirmed through kariotyping. Multiple screening tests may be used to test or diagnosis a patient prior to birth (biomarkers, nuchal translucency, amniocentesis, etc.). A Down's syndrome patient may be diagnosed with AD using diagnostic criteria relevant for AD.

In numerous embodiments a method includes identifying a subject suffering from or at risk of Lewy body dementia and removing Aβ or another target molecule from the patient's CSF via apheresis. Those suffering from or at risk of Lewy body dementia can be identified by mental, psychiatric or neuropyschological assessments, blood tests, brain imaging (PET, MRI, CT scan), urine tests, tests on the cerebrospinal fluid obtained through lumbar puncture, or the like. Those at risk of Lewy body dementia include those of advancing age.

In various embodiments, cerebral plaques may be cleared or prevented from forming by removing Aβ or another target molecule from the patient's CSF via apheresis. It will be understood that achieving any level of clearing of a plaque or plaques will constitute clearing of the plaque or plaques. It will be further understood that achieving any level of prevention of formation of a plaque or plaques will constitute preventing formation of the plaque or plaques. The methods may further include clearing or preventing parenchymal amyloid plaques or soluble forms of Aβ. The methods may further include improving cognitive aspects of the subject.

In some embodiments, cognitive abilities of a subject are improved by removing Aβ or another target molecule from the patient's CSF via apheresis.

In various embodiments, parenchymal amyloid plaques or soluble forms of Aβ are cleared in a subject by removing Aβ or another target molecule from the patient's CSF via apheresis.

The ability of a therapy described herein to treat a disease may be evaluated through medical examination, e.g. as discussed above, or by diagnostic or other tests. In various embodiments, a method as described in WO 2006/107814 (Bateman et al.) is performed. For example, a subject may be administered radiolabeled leucine. Samples, such as plasma or CSF, may then be obtained to quantify the labeled-to-unlabeled leucine in, for example, amyloid beta or other key disease related biomarkers, to determine the production and clearance rate of such proteins or polypeptides.

Clearing of, or formation of, amyloid beta can be evaluated in vivo by structural or functional neuro-imaging techniques. For example, diffusion tensor MRI (reviewed in Parente et al., 2008; Chua et al., 2008), PET imaging with the Aβ binding compound, Pittsburgh Compound B (PiB, Klunk et al., 2004; Fagan et al., 2006; Fagan et al 2007) or other SPECT based imaging of fibrillar Aβ structures and measurement of CSF levels of Aβ42 or tau may be employed. Distribution of vascular Aβ may be evaluated using differential interpretation of PET imaging of PiB (Johnson et al., 2007). Additionally, a cerebral microhemorrahage may be recognized by on gradient-echo or T-2 weighted MRI sequences (Viswanathan and Chabriat, 2006).

Similarly, detection of hemorrhages of the cerebral vasculature can be evaluated by imaging techniques, clinical evaluation, or the like. Spontaneous intracerebral hemorrhage (ICH) usually results in a focal neurologic deficit and is easily diagnosed on clinical and radiographic grounds (computed tomography (CT) scan, T-2 weighted MRI). Cerebral microhemorrhage results from underlying small vessel pathologies such as hypertensive vasculpathy or CAA. Cerebral microhemorrhages, best visualized by MRI, result from rupture of small blood vessels. The MRI diagnosis can be variable as described by Orgagozo et al., 2003 (Subacute meningoencephalitis in a subset of patients with AD after Aβ42 immunization-Elan Trial). For instance, patients showing signs and symptoms of aseptic meningoencephalitis MRIs showed only meningeal enhancement, whereas others had meningeal thickening, white matter lesions, with or without enhancing or edema, and a majority had posterior cerebral cortical or cerebellar lesions. Other potential diagnostics include changes in intracranial pressure, which may be detected by specific MRI techniques (Glick, et al., 2006, Alperin) or other standard techniques as described in Method of detecting brain microhemmorhage (U.S. Pat. No. 5,951,476).

### Combined Apheresis and Administration of Agent

In addition to apheresis as discussed above, one or more therapeutic agents may be administered to a subject for purposes of treatment or study. For example, a therapeutic agent may be administered in combination with apheresis to study the combined effects of apheresis and the agent on a subject at a molecular level, a pharmacological level, at a physiological level, at a behavioral level, or the like. Any one or more condition, symptom, or disease may be treated. For example, the diseases discussed above, such as Alzheimer's disease (AD), cerebral amyloid angiopathy (CAA), Lewy body dementia, Down's syndrome (DS), and Parkinson's disease (PD) may be treated or studied.

Any suitable therapeutic agent may be administered for purposes of investigation or treatment. Suitable agents include small molecule chemical compounds, antibodies, inhibitory polynucleotides, expression vectors, or the like. In some embodiments, it may be desirable to conjugate the therapeutic agent with a molecule capable of enhancing uptake of the therapeutic agent into cells, particularly when the therapeutic agent is a large molecule. Conjugation may be done according to any known or future developed technique with any known or future developed conjugate. One example is the conjugation of polypeptides with mannose, e.g. as described US Patent Publication No. 2005/0208090. US Patent Publication No. 2005/0208090 also describes various other components to compositions that may be useful for delivering molecules to the CNS of a subject.

If the therapeutic agent is a polypeptide or polynucleic acid, sequences may readily be obtained by those of skill in the art. For example, the GenBank database or other similar databases may be searched to obtain sequences of proteins or genes of interest. If the therapeutic agent is a polynucleic acid configured to serve as gene therapy for the purposes of investigation or study, the polynucleic acid will be present in an expression vector. Voluminous publications, including published patent applications and patents, describe how to effectively produce expression vectors, and thus are not described herein in detail. If the therapeutic agent is a polynucleic acid configured to serve as an inhibitory polynucleic acid configured to suppress expression of a target gene, the polynucleic acid will be present in a suitable form, such as short interfering nucleic acid (siNA), short interfering RNA (siRNA), double-stranded RNA (dsRNA), micro-RNA (miRNA), short hairpin RNA (shRNA), or the like. A detailed description of suitable forms of such inhibitory polynucleic acids are described in numerous publications, including published patent applications and patents and thus are not described herein in great detail. One example of a patent publication providing a detailed description of inhibitory polynucleotudes is U.S. Patent Application Publication Number 20070270579.

Agents may be administered via any suitable route, such an intrathecal, intracerebroventricular, or the like. In some embodiments, the therapeutic agent is administered to the subject's CSF along with apheresed CSF fluid that is returned to the patient.

Therapeutic agents can be administered in the form of pharmaceutical compositions. Such pharmaceutical compositions can include the therapeutic and one or more other pharmaceutically acceptable components. See Remington's Pharmaceutical Science (15th ed., Mack Publishing Company, Easton, Pa. (1980)). The preferred form depends on the intended mode of administration and therapeutic application. The compositions may also include, depending on the formulation desired, pharmaceutically-acceptable, non-toxic carriers or diluents, which are defined as vehicles commonly used to formulate pharmaceutical compositions for animal or human administration. The diluent is selected so as not to adversely affect the biological activity of the therapeutic agent. Examples of such diluents are distilled water, physiological phosphate-buffered saline, artificial cerebrospinal fluid, citrate buffered saline, Ringer's solutions, dextrose solution, and Hank's solution. In addition, the pharmaceutical composition or formulation may also include other carriers, adjuvants, or nontoxic, non-therapeutic, non-immunogenic stabilizers or the like.

Pharmaceutical compositions can also include large, slowly metabolized macromolecules such as proteins, polysaccharides such as chitosan, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i.e., adjuvants).

In various embodiments, the compositions are formulated as injectable compositions. Injectable compositions include solutions, suspensions, dispersions, or the like. Injectable solutions, suspensions, dispersions, or the like may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Injectable compositions that include a therapeutic agent may be prepared in water, saline, isotonic saline, phosphate-buffered saline, citrate-buffered saline, or the like and may optionally be mixed with a nontoxic surfactant. Dispersions may also be prepared in glycerol, mannitol, liquid polyethylene, glycols, DNA, vegetable oils, triacetin, or the like or mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols or the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, or the like. Isotonic agents such as sugars, buffers, or sodium chloride may be included. Prolonged absorption of the injectable compositions can be brought about by the inclusion in the composition of agents delaying absorption; e.g., aluminum monosterate hydrogels or gelatin. Solubility enhancers may be added. Sterile injectable compositions may be prepared by incorporating a therapeutic agent in the desired amount in the appropriate solvent with various other ingredients, e.g. as enumerated above, and followed by sterilization, as desired, by, for example, filter sterilization. In the case of sterile powders for the preparation of sterile injectable solutions, methods of preparation include vacuum drying and freeze-drying techniques, which yield a powder of the active ingredient plus any additional desired ingredient present in a previously sterile-filtered solution.

For prolonged delivery of a fluid composition to a subject, it may be desirable for the composition to be isotonic with the tissue into which the composition is being delivered. For example, the fluid composition may be isotonic with a subject's CSF. CSF typically has a tonicity of about 305 mOsm. Accordingly, fluid compositions intended for delivery to CSF may advantageously have a tonicity of about 290 mOsm to about 320 mOsm. If during formulation the composition has a tonicity lower than about 290 mOsm to about 320 mOsm, the tonicity may be enhanced by adding a tonicity enhancing agent, such as sodium chloride. As used herein, “tonicity enhancing agent” means a compound or composition that increases tonicity of a composition. However, such tonicities of between about 290 mOsm to about 320 mOsm are not always achievable. When the concentration of therapeutic agent in a fluid composition renders the composition hypertonic relative to a subject's physiological fluid, it is preferred that little or no amount of a tonicity enhancing agent be added to the composition. However, it will be recognized that it may desirable to add one or more additional compounds to the composition even though the addition of the additional compound(s) will further increase tonicity of the composition. For example, it may be desirable to add to the composition an additional therapeutic agent, stabilizing compound, preservative, solubilizing agent, buffer, etc., even though tonicity will be increased.

In various embodiments, the final solution is adjusted to have a pH between about 4 and about 9, between about 5 and about 7, between about 5.5 and about 6.5, or about 6. The pH of the composition may be adjusted with a pharmacologically acceptable acid, base or buffer. Hydrochloric acid is an example of a suitable acid, and sodium hydroxide is an example of a suitable base. The hydrochloric acid or sodium hydroxide may be in any suitable form, such as a 1N solution.

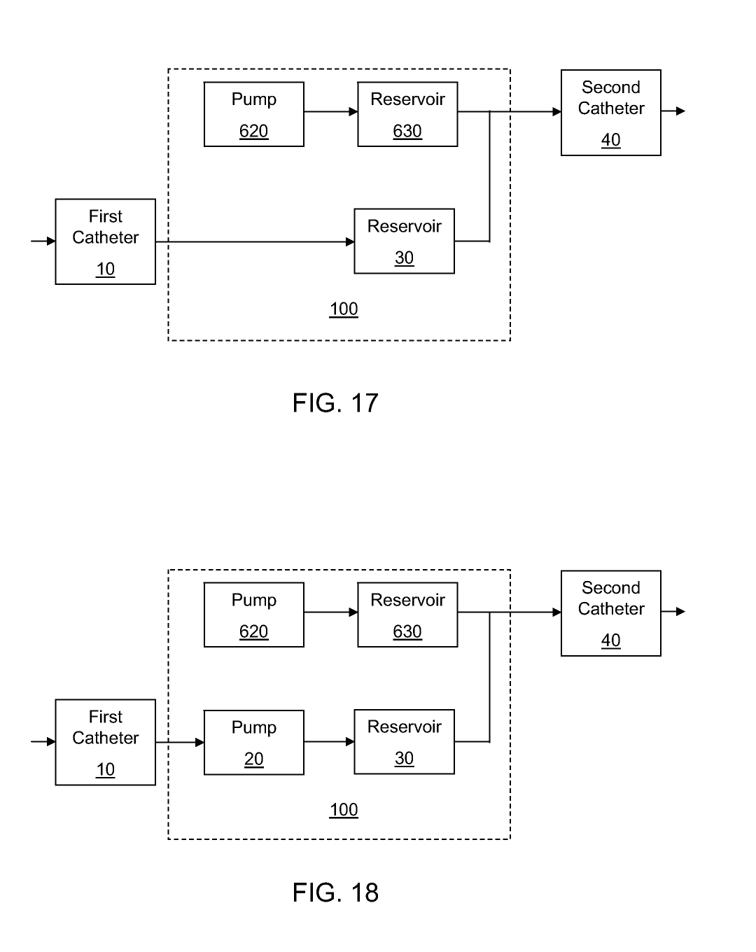

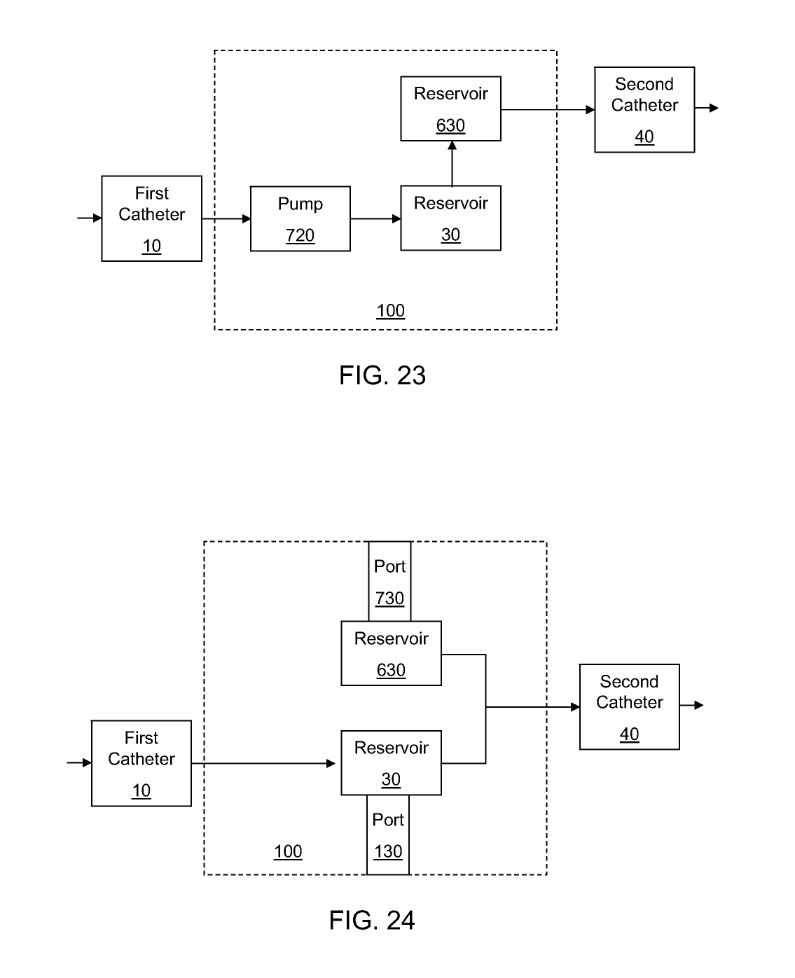

It will be understood that the concentration of therapeutic agent in a pharmaceutical composition may vary based on a variety of factors, including the solubility of the therapeutic agent itself. When delivered via an implantable infusion system (e.g., as discussed in more detail below with regard to FIGS. 17-29), a high concentration of therapeutic agent may be desirable, so that the agent may be delivered over an extended period of time to avoid frequent refills of the infusion device. In various embodiments, the therapeutic agent is an antibody present in an injectable therapeutic composition at a concentration of between about 0.001 mg/ml and about 50 mg/ml (e.g, between about 0.1 mg/ml and about 10 mg/ml).

A therapeutic agent may be administered at any suitable dose. It will be understood that the dose of the therapeutic agent may vary depending on the therapeutic agent used, the condition to be treated or studied, the effect on the subject, whether more than one therapeutic agent is administered, or the like. For agents that have previously been investigated or used clinically, one of skill in the art will be readily able to determine a suitable dosage. Suitable dosages can readily be extrapolated from in vitro studies by comparing to agents tested in vivo and having similar mechanisms of action.

In various embodiments, an agent is delivered into a subject with CSF from which a target molecule has been removed via apheresis. The agent may be delivered via any suitable mechanism. By way of example, an agent may be infused via a port 130 as in, e.g., FIGS. 5-6; port 300 as shown in, e.g., FIG. 7; port 210, 220 as shown in, e.g., FIG. 13; port 130 as shown in, e.g., FIGS. 14-15; or the like. Further examples of suitable catheters and delivery systems for administering an agent to the brain (intraparenchymally or intracerebroventricularly) are described in WO 2008/054700, entitles INFUSION CATHETERS, published on May 8, 2008, and assigned to Medtronic, Inc. In some embodiments a device includes a reservoir for housing a therapeutic agent and a separate reservoir for housing media for removing a target molecule via apheresis.

For example and with reference to FIGS. 17-23, block diagrams of some representative components of examples of various device 100 configurations are shown. It will be understood that the devices shown in FIGS. 17-22 may contain other components that are not shown, such as valves, refill or access ports, processors, power supplies, or the like. The devices 100 in the embodiments depicted in FIGS. 17-22 include a first reservoir 30 and a second reservoir 630. The first reservoir 30 is configured to house media for removing a target molecule from CSF and may be as described above with regard to, e.g., FIGS. 8-15. The second reservoir 630 is configured to house a fluid composition containing a therapeutic agent. The device 100 may include one or more pumps 20, 620 for pumping CSF through reservoir 30 or therapeutic agent from reservoir 630.

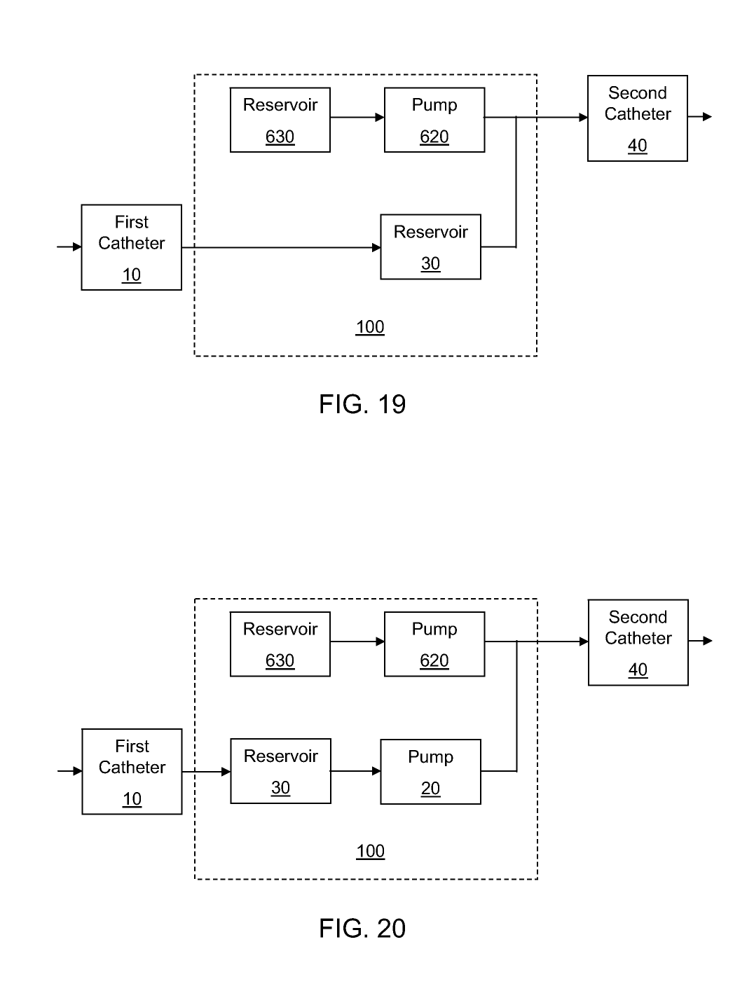

The devices 100 shown in FIG. 17 and FIG. 19 do not include a pumping mechanism for apheresis. CSF enters the reservoir 30 containing media for removing a target molecule via first catheter 10 and exits the device and is returned to a CSF compartment via second catheter 40. Therapeutic agent is pumped from the device 100 and is delivered to the CSF compartment via the second catheter 40. A pump 620 may be upstream (FIG. 17) or downstream (FIG. 19) of the reservoir 630 housing the therapeutic agent, depending on the pump 620 configuration. Pump 620 may be any suitable pump. For example, the pump 620 may be a peristaltic pump, an osmotic pump, a piston pump, a diaphragm pump, or the like. The pump 620 may be fixed rate, variable rate, programmable, etc.

In the embodiments depicted in FIG. 18 and FIG. 20, device includes two pumps 20, 620. One pump 20 is operably coupled to the reservoir 30 containing media for removal of a target molecule from CSF via apheresis. The other pump 620 is operably coupled to the reservoir 630 housing the therapeutic agent. Some configurations with the pumps 20, 620 upstream or downstream of the reservoirs 30, 630 are shown. Other configurations of pumps relative to reservoirs are readily envisioned and are contemplated herein.

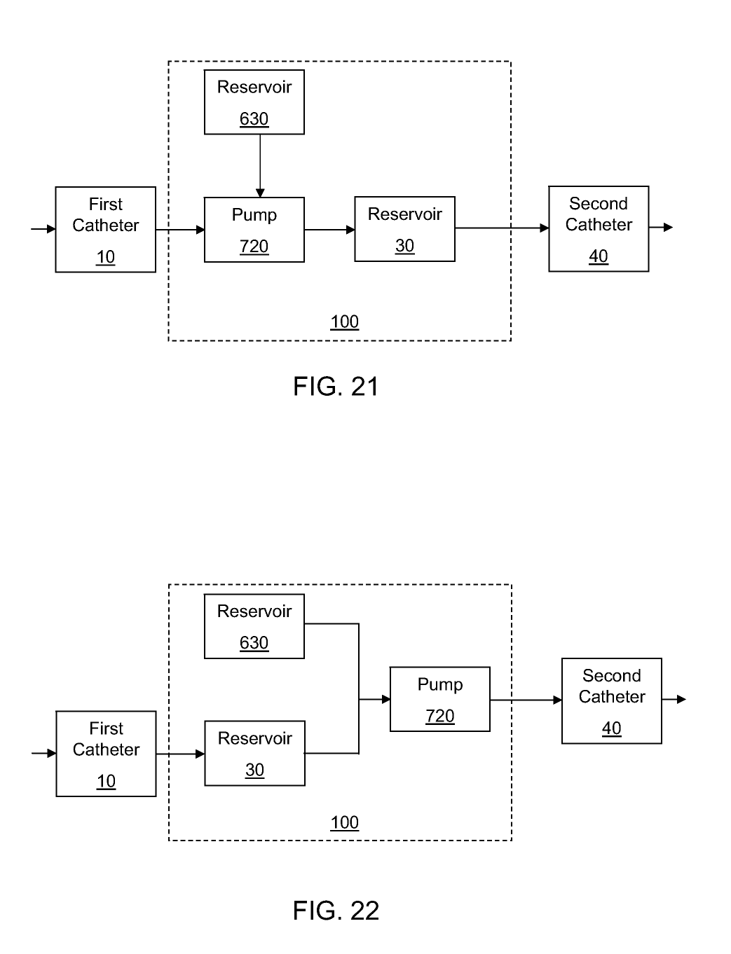

In the embodiments depicted in FIGS. 21-23, the device 100 includes one pump 720 that is operably coupled to both reservoirs 30, 630. Various configurations of relative placement (upstream or down stream) of reservoir 30, reservoir 630, and pump 720 are shown in FIGS. 21-23. Other relative configurations of pumps and reservoirs are readily envisioned and are contemplated herein. Pump 720, like pump 620 and pump 20 discussed above, may be any suitable pump, such as a peristaltic pump, an osmotic pump, a piston pump, a diaphragm pump, or the like. Pump 720 may be fixed rate, variable rate, programmable, etc. While not shown, it will be understood that it may be desirable to include one or more valves or flow restrictors to control the relative flow rate of fluid from the first 30 and second 630 reservoirs. The valves may be controlled via a processor (not shown) or other similar component.

Referring now to FIG. 24, a device 100 may include access ports 130, 730 for infusing or withdrawing fluid from reservoirs 30, 630. For example, access port 130 may provide access to reservoir 30 so that the medium for removing a target molecule may be replaced or regenerated. Access port 730 may provide access to reservoir 630 to fill or refill reservoir 630 with fluid containing therapeutic agent, to sample fluid from the reservoir 630 to determine the stability of the agent, or the like.

It will be understood that the device configurations shown in FIGS. 17-24 are shown as examples of configurations that may be employed and that other configurations are possible. It will also be understood that components that are not shown in a given embodiment may be included in such embodiments.

### Exemplary Agents for Treatment or Investigation of Alzheimer's Disease

For purposes of example, some exemplary therapeutic agents or classes of agents, which may be useful for treatment or study of Alzheimer's disease (AD), other dementias, or diseases associated with accumulation of amyloid beta, are discussed below. Any reference herein to a particulate therapeutic agent includes the agent and pharmaceutically acceptable salts, solvates, hydrates and polymorphs thereof.

Examples of therapeutic agents that may be used alone or in combination, along with apheresis of a target molecule, for treatment of AD, other dementias, or diseases associated with accumulation of amyloid beta include: (i) ApoE-modulating agents; (ii) agents that directly or indirectly inhibit the function of the receptor for advanced glycation end products (RAGE); (iii) β-secretase 1 (BACE1) inhibitors; (iv) γ-secretase inhibitors; (v) muscarinic receptor subtype 1 (M1) agonists; (vi) growth factors; (vii) enzymes capable of degrading amyloid beta; (viii) mitochondrial antioxidants; (ix) insulin, with or without insulin-sensitizing agents; (x) microtubule stabilizing agents; and (xi) inhibitors of tumor necrosis factor (TNF).

i. ApoE-Modulating Agents

A variety of ApoE-modulating agents may be beneficial for treating or investigating AD, other dementias, or diseases associated with accumulation of amyloid beta, particularly in subjects carrying at least one allele of ApoE-ε4. Some therapeutic approaches associated with ApoE are described in Bu (2009) “Apolipoprotein E and its receptors in Alzheimer's disease: pathways, pathogenesis and therapy”, Nature Review Neuroscience 10, 333-344. By way of example, agents that (i) convert ApoE4 to ApoE3 (e.g. by disrupting ApoE4's domain interaction), (ii) increase ApoE levels, such as liver X receptor (LXR) agonists, (iii) mimic ApoE, such as peptidomimmics, (iv) block APOE-amyloid beta interaction, such as amyloid beta 12-28, (v) block ApoE fragmentation, (vi) increase ApoE lipidation, (vii) increase LRP1 or LDLR level, or (viii) increase APOER2 or VLDLR level may be beneficial.

LXR agonists may be of particular interest due to their commercial availability. Oxysterols, the oxygenated derivatives of cholesterol, such as 22(R)-hydroxycholesterol, 24(S)-hydroxycholesterol, 27-hydroxycholesterol, and cholestenoic acid are natural LXR ligands that may be employed. Examples of synthetic LXR agonists include T0901317 and GW3965. LXR agonists may produce unwanted peripheral side effects involving lipid metabolism, and thus may be good candidates for central administration, such as administration to a CSF compartment with apheresed CSF.

ii. RAGE Inhibtors

Molecules that directly or indirectly inhibit the function of the receptor for advanced glycation end products (RAGE) may be beneficial for treating or investigating AD, other dementias, or diseases associated with accumulation of amyloid beta. Interaction between RAGE and amyloid beta can result in neuronal cell death by oxidative damage or microglial activation. One RAGE inhibitor that may be desirably employed in accordance with the teachings presented herein is PF-04494700 (formerly known as TTP488). The RAGE gene may also be targeted; e.g. via RNA-mediated inhibition or silencing.

iii. BACE1 inhibitors

Any suitable BACE1 inhibitor may be administered to a subject for purposes of treatment or investigation. A number or BACE1 inhibitors are known and include transition-state analogs and peptide-mimetic inhibitors, hydroxyethylamine-based compounds, other isosteres, heterocyclic inhibitors, and other non-peptidic inhibitors, as reviewed in Evin and Kenvhe (2008) “BACE1 Inhibitors” Recent Patents on CNS Drug Discovery 2(3):188-199. Examples of particular BACE1 inhibitors include the APP analog STA-200, the APP analog OM99-2, the APP analog OM00-3, the APP analog KMI-429, GSK-188909, pyrrolidine, piperidine, macrocyclic piperazin-2-one, 2-alkoxyl morpholine, macrocyclic isopthalamide, acylguanidine, 2-amino-quinazoline, pyrimidone, spiropiperidine, tetronic acid, and a cumarin derivative developed by Kraus/INSERM, each of which is discussed in the Evin and Kenvhe review. CNS-targeted delivery of BACE1 inhibitors may provide an opportunity for establishing and maintaining therapeutically effective concentrations of the compound in the brain before its efflux by P-glycoprotein and may also reduce the potential for side effects based on inhibition of targets other than BACE1.

In various embodiments, the BACE1 gene is targeted; e.g. via RNA-mediated inhibiting or silencing.

### iv. γ-Secretase Inhibitors

Any suitable γ-secretase inhibitor may be administered to a subject for purposes of treatment or investigation. Examples of γ-secretase that may be beneficially used include, among others, R-Flurbiprofen (Myriad genetics), MCP-7869, LY-450139 (Eli Lilly&Co), LY411575 (Eli Lilly&Co), and MK0752 (Merck Inc). CNS-targeted delivery of γ-secretase inhibitors may provide an opportunity for establishing and maintaining therapeutically effective concentrations of the compound in the brain and may also reduce the potential for side effects based on inhibition of targets other than γ-secretase.

In some embodiments, expression of the XII gene (APAB) is silenced or reduced. Any suitable method for silencing or reducing expression of the X11 gene may be used. For example, RNA interference as described in Xie et al. (2005) “RNA Interference-mediated Silencing of X1-alpha and X11-beta Attenuates Amyloid beta-Protein Levels via Differential Effects on beta-Amyloid Precursor Protein Processing” J. Biol. Chem. 15, 15413-15421 may be employed. Other gene targets that may also lead to selective regulation of γ-secretase activity such as presenilin, PEN-2, APH-1, nicastrin and TMP21.

v. M1 Agonists

Any suitable M1 agonist may be administered to a subject for purposes of treatment or investigation. Preferably, the M1 agonist is selective for the M1 receptor. However, if administered directly to the CNS, e.g. by administering with apheresed CSF to a CSF compartment, peripheral side effects associated with less selective muscarinic agonists may be reduced. Examples of M1 agonists having some selectivity towards the M1 receptor include cevimeline, talsaclidine, sabcomeline and milameline. Xanomeline is an example of a more selective M1 agonist that may be used. A number of 1,4,5,6-tetrahydropyrimidine moiety-containing M1 agonists have been developed and are described in the literature. 5-(3-ethyl-1,2,4-oxadiazol-5-yl)-1,4,5,6-tetrahydropyrimmidine (CDD-0102) is one such M1 agonist with high potency and a low side-effect profile.

vi. Growth Factors

One or more suitable growth factors may be beneficially administered for treating or investigating AD, other dementias, or diseases associated with accumulation of amyloid beta. As used herein, “growth factor” refers to isolated or recombinant forms of endogenous growth factors and functional variants thereof “Functional variants” refers to variants that are agonists of the receptor for the endogenous growth factor. Functional variants may have any suitable degree of sequence identity; e.g., more than 70% sequence identity, more than 80% sequence identity, more than 90% sequence identity, more than 95% sequence identity, more than 98% sequence identity, or more than 99% sequence identity, to the endogenous growth factor.

Examples of suitable growth factors that may be administered include vascular endothelial growth factor (VEGF), brain-derived neurotrophic factor (BDNF), nerve growth factor (NGF) and related growth factors, and insulin-like growth factor (IGF), particularly IGF-1. Recombinant human VEGF is available from R&D systems. Clinical grade recombinant growth factors are also available as an API (active pharmaceutical ingredient).

vii. Enzymes Capable of Degrading Amyloid Beta

Any suitable agent capable of degrading amyloid beta may be used in accordance with the teachings presented herein. Neprilysin is an example of an enzyme capable of degrading amyloid beta, a recombinant human form of which is available from R&D systems. Other enzymes capable of degrading amyloid beta include insulin-degrading enzyme (IDE), angiotensin-converting enzyme (ACE), and insulysin. Direct delivery of such amyloid beta degrading enzymes to the CNS should improve their safety and efficacy. Because such enzymes act in several pathways, careful dose titration on a patient-by-patient basis may be desired when used for therapeutic purposes.

viii. Mitochondrial Antioxidants

Any suitable mitochondrial antioxidant may be beneficially administered for treating or investigating AD, other dementias, or diseases associated with accumulation of amyloid beta. A number of mitochondrial antioxidants are known and include those compounds disclosed in U.S. Pat. No. 6,417,220, entitled Mitochondrial membrane stabilizers, issued on Jul. 9, 2002 to Yoshii et al, and include those compounds discussed in Yamada and Harashima (2008) “Mitochondrial drug delivery systems for macromolecule and their therapeutic application to mitochondrial diseases” Adv. Drug Deliv. Resv. 60(13-14):1439-62. Although the numerous small molecule approaches to achieve mitochonodrial stabilization generally cross the blood brain barrier, CNS targeted delivery of these agents may improve their safety and efficacy. Because these compounds target a cellular function that is essential and ubiquitous to all cells it is likely that this approach will have a narrow therapeutic window. Accordingly, careful dose titration on a patient-by-patient basis may be desired when used for therapeutic purposes.

ix. Insulin

Any suitable form of insulin may be administered for the purposes of therapy or investigation. Preferably, insulin is administered directly to the CNS, e.g. along with apheresed CSF to a CSF compartment of a subject. Such CNS-targeted delivery would provide the opportunity to titrate the appropriate dose per patient based on comorbidities (such as diabetes, hypoglycemia and cardiovascular disease) while avoiding systemic exposure, which may worsen memory and cognition in some AD patients, and unwanted effects on metabolic and endocrine system.

In various embodiments, insulin is administered in combination with an insulin-sensitizing agent, such as glitazones (pioglitazone and rosiglitazone and other peroxisome proliferator-activated receptor (PPAR) gamma agonists) that reduce insulin resistance.

x. Microtubule Stabilizing Agents

Any suitable microtubule stabilizing agent may be administered in accordance with the teachings presented herein. For examples kinase inhibitors that decrease phosphorylation of Tau, autophagy stimulators, such as inhibitors of mTOR kinase, and other microtubule stabilizing agents may be beneficially used. The microtubule stabilizing compound peptide NAP(AL-108 Allon Therapeutics) is an example of a microtubule stabilizing agent that is fairly well advanced in the clinic.

xi. Inhibitors of TNF

Any suitable inhibitor of TNF may be administered to a subject in accordance with the teachings presented herein. Inhibitors of TNF include small molecule chemical agents and biological agents, such as polynucleotides and polypeptides, which include antibodies and fragments thereof, antisense, small interfering RNA (siRNA), and ribosymes. Examples of inhibitors of TNF include soluble TNF inhibitors, such as fusion proteins (such as etanercept); monoclonal antibodies (such as infliximab and D2E7); binding proteins (such as onercept); antibody fragments (such as CDP 870); CDP571 (a humanized monoclonal anti-TNF-alpha IgG4 antibody), soluble TNF receptor Type I, pegylated soluble TNF receptor Type I (PEGs TNF-R1) and dominant-negative TNF variants, such as DN-TNF and including those described by Steed et al. (2003), “Inactivation of TNF signaling by rationally designed dominant-negative TNF variants”, Science, 301 (5641): 1895-8.

xii. Anti-Amyloid Beta Antibodies

Any suitable anti-amyloid beta antibody may be administered to a subject in accordance with the teachings presented herein. The term “antibody” is used in the broadest sense and specifically includes, for example, single anti-Aβ monoclonal antibodies (including agonist, antagonist, and neutralizing antibodies), anti-Aβ antibody compositions with polyepitopic specificity, single chain anti-anti-Aβ antibodies, and fragments of anti-Aβ antibodies. In various embodiments, the antibodies are humanized antibodies. In various embodiments, an anti-amyloid antibody is an antibody as described in US Patent Application Publication No. 2009/075923 (application Ser. No. 12/323,682), entitled HUMANIZED ANTI-AMYLOID BETA ANTIBODIES, published on Jul. 9, 2009.

xiii. Other Agents

Any other suitable agent may be beneficially administered to a subject for treating or investigating AD, other dementias, or diseases associated with accumulation of amyloid beta. For example, clioquinol, alzhemed, or other agents that target important binding sites on amyloid beta, such as compounds that target metal binding sites or glycosaminoglycan (GAG) sites, may be used. Gelsolin, GM1 ganglioside, agents that increase the function or expression of LRP, such as soluble LRP, plasmin, and scyllo-cyclohexanehexyl AZD-103 (Transition therapeutics) or other agents that target disaggregation of amyloid beta are examples of other agents that may be beneficially administered for purposes of treatment or investigation in accordance with the teachings presented herein.

### Direct Delivery of Agents to the CNS

In various embodiments, agents may be delivered directly to the central nervous system (CNS) of a subject without concomitant apheresis. The agents may be administered in any suitable manner, such as intrathecally, intracerebroventricularly, or intraparenchymally. The agents may be useful for reducing CNS levels of amyloid beta or for treating Alzheimer's disease or other dementias. In various embodiments, the agents are selected from the group consisting of an ApoE-modulating agent; a RAGE inhibitor; a β-secretase 1 (BACE1) inhibitor; a γ-secretase inhibitor; a muscarinic receptor subtype 1 (M1) agonists; a growth factor; an enzyme capable of degrading amyloid beta; a mitochondrial antioxidant; insulin; an inhibitor of tumor necrosis factor (TNF), and an anti-amyloid beta antibody. The agents, devices, systems and associated methods described herein may be used to treat or study a disease associated with amyloid beta accumulation in a subject, such as Alzheimer's disease; e.g. as discussed above.

By delivering the agents directly to a CSF compartment of a subject, peripheral side effects may be reduced and agents that may not be able to cross the blood-brain barrier in sufficient quantities may be used. Other advantages associated with the systems, devices and methods described herein will be readily evident to those of ordinary skill in the art.

In various embodiments, formulations containing a therapeutic agent for reducing amyloid beta or treating Alzheimer's disease or other dementias are administered to the CNS via an infusion device. According to various embodiments, a composition comprising a therapeutic agent is delivered directly to cerebrospinal fluid of a subject. A composition containing a therapeutic agent may be delivered to cerebrospinal fluid of a subject anywhere that the cerebrospinal fluid is accessible. For example, the composition may be administered intrathecally, intracerebroventricularly, e.g. via the lateral ventricle or third ventricle, or into the subarachnoid space over the cortical convexities of the brain.

In some embodiments, a composition containing a therapeutic agent is administered directly into brain tissue of a subject (intraparenchymally).

Any suitable delivery device or system may be employed to deliver a therapeutic agent directly to the CNS of a subject. For example, implantable or external infusion devices may be employed. In some embodiments an implantable infusion device having a fluid drive mechanism is employed. In some embodiments, a system employing an implantable port operably coupled to a catheter is employed, where the port is accessible via a hypodermic needle or cannula from outside the subject.

If a system includes a drive mechanism, whether implantable or external, any suitable drive mechanism may be employed. Non-limiting examples of drive mechanisms include peristaltic pumps, osmotic pumps, piston pumps, pressurized gas mechanisms, and the like. Devices including such drive mechanisms may be fixed-rate pumps, variable rate pumps, selectable rate pumps, programmable pumps and the like. For the purposes of this disclosure and the appended claims, “drive mechanism” and “pump” are used interchangeably. Infusion systems or devices employing a pump also include a reservoir for housing a fluid composition containing the therapeutic agent. A catheter may be operably coupled to the reservoir and may be used to deliver the therapeutic fluid to one or more target locations of the subject.

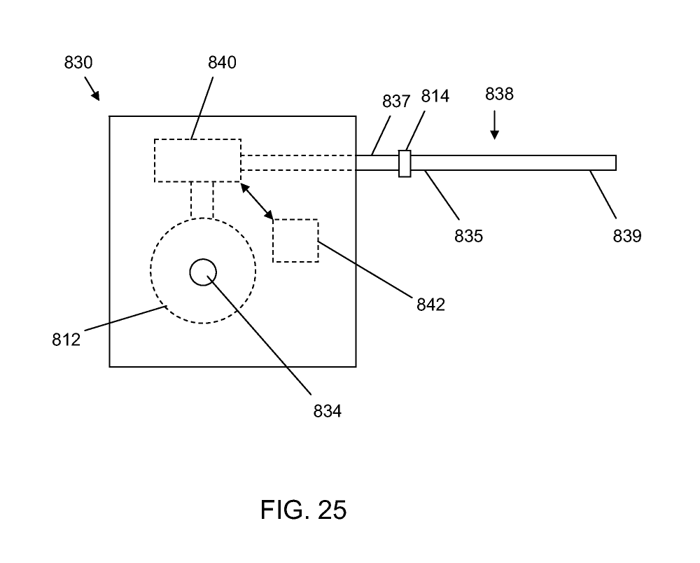

Non-limiting embodiments of infusion systems and devices, or representative components thereof, are illustrated in FIGS. 25-28.

Referring now to FIG. 25, an example of a suitable implantable system is shown, where internal components are depicted in dashed lines. The system includes an infusion device 830 and a catheter 838. The infusion device 830 includes a reservoir 812 for housing a composition and a drive mechanism 840 operably coupled to the reservoir 812. The catheter 838 shown in FIG. 1 has a proximal end 835 coupled to the therapy delivery device 830 and a distal end 839 configured to be implanted in a target location of a subject. Between the proximal end 835 and distal end 839 or at the distal end 839, the catheter 38 has one or more delivery regions (not shown), such as openings, through which the composition may be delivered. The infusion device 830 may have a port 834 into which a hypodermic needle can be inserted to inject a composition into reservoir 812. The infusion device 830 may have a catheter port 837, to which the proximal end 835 of catheter 838 may be coupled. The catheter port 837 may be operably coupled to reservoir 812. A connector 814, such as a barbed connector or sutureless connector, may be used to couple the catheter 838 to the catheter port 837 of the infusion device 830. The infusion device 830 may be operated to discharge a predetermined dosage of the pumped fluid into a target region of a subject. The infusion device 830 may contain a microprocessor 842 or similar device that can be programmed to control the amount of fluid delivery. The programming may be accomplished with an external programmer/control unit via telemetry. A controlled amount of fluid may be delivered over a specified time period. With the use of a programmable infusion device 830, dosage regimens may be programmed and tailored for a particular patient. Additionally, different therapeutic dosages can be programmed for different combinations of fluid comprising therapeutics. Those skilled in the art will recognize that a programmable infusion device 830 allows for starting conservatively with lower doses and adjusting to a more aggressive dosing scheme, if warranted, based on safety and efficacy factors.

While not shown in FIG. 25, device 830 may include a catheter access port to allow for direct delivery of a composition including a therapeutic agent via catheter 838. Also not shown are other components, such as one-way valves, that may be included at one or more locations along the fluid flow path of the device 830, a power supply to drive operation of the processor 842 or drive mechanism 840, etc. It will be understood that the components and the configuration of the components depicted in FIG. 25 may be readily modified to achieve a suitable infusion device 830 for delivering an injectable composition including a therapeutic agent for reducing amyloid beta or for treating Alzheimer's disease or other dementias.

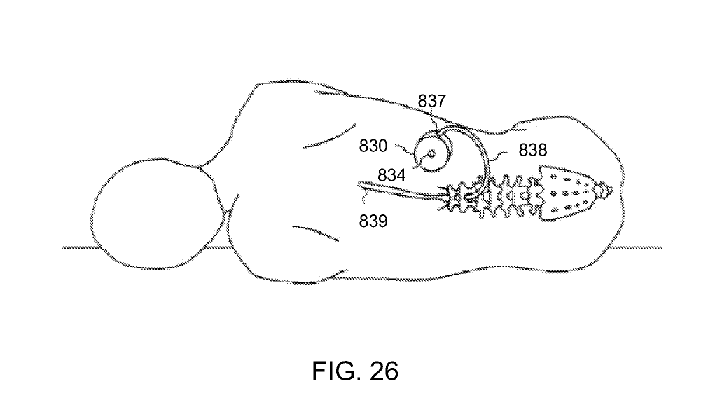

FIG. 26 illustrates a representative implantable system configured for intrathecal delivery of a composition containing a therapeutic agent. As shown in FIG. 26, a system or device 830 may be implanted below the skin of a patient. Preferably the device 830 is implanted in a location where the implantation interferes as little as practicable with activity of the subject in which it is implanted. One suitable location for implanting the device 830 is subcutaneously in the lower abdomen. In various embodiments, catheter 838 is positioned so that the distal end 839 of catheter 838 is located in the subarachnoid space of the spinal cord such that a delivery region (not shown) of catheter is also located within the subarachnoid space. It will be understood that the delivery region can be placed in a multitude of locations to direct delivery of an agent to a multitude of locations within the cerebrospinal fluid of the patient. The location of the distal end 839 and delivery region(s) of the catheter 838 may be adjusted to improve therapeutic efficacy.

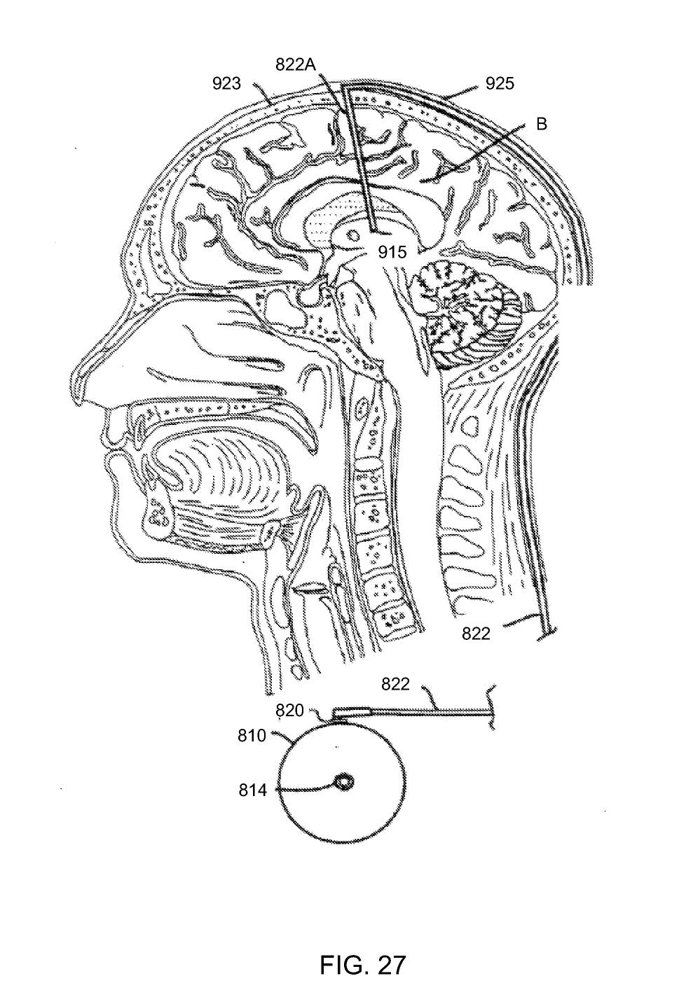

According to various embodiments, a composition containing a therapeutic agent is delivered intraparenchymally directly to brain tissue of a subject. In some embodiments, the composition is delivered to a subject's hippocampus, formix (e.g., any portion of the circle of Papez), or cortical loci such as the entorhinal cortex. An infusion device may be used to deliver the agent to the brain tissue. A catheter may be operably coupled to the infusion device and a delivery region of the catheter may be placed in or near a target region of the brain. One suitable system for administering a therapeutic agent to the brain is discussed in U.S. Pat. No. 5,711,316 (Elsberry). Referring to FIG. 27, a system or infusion device 810 may be implanted below the skin of a subject. The device 810 may have a port 814 into which a hypodermic needle can be inserted through the skin to inject a quantity of a composition comprising a therapeutic agent. The composition is delivered from device 810 through a catheter port 820 into a catheter 822. Catheter 822 is positioned to deliver the agent to specific infusion sites in a brain (B). Device 810 may take the form of the like-numbered device shown in U.S. Pat. No. 4,692,147 (Duggan), assigned to Medtronic, Inc., Minneapolis, Minn., or take the form of a SYNCHROMED II® (Medtronic, Inc.) infusion device. The distal end of catheter 822 terminates in a cylindrical hollow tube 822A having a distal end 915 implanted into a target portion of the brain by conventional stereotactic surgical techniques. Additional details about end 915, according to various embodiments, may be obtained from U.S. application Ser. No. 08/430,960 entitled “Intraparenchymal Infusion Catheter System,” filed Apr. 28, 1995 in the name of Dennis Elsberry et al. and assigned to the same assignee as the present application. Tube 822A is surgically implanted through a hole in the skull 923 and catheter 822 is implanted between the skull and the scalp 925 as shown in FIG. 27. Catheter 822 may be coupled to implanted device 810 in the manner shown or in any other suitable manner.

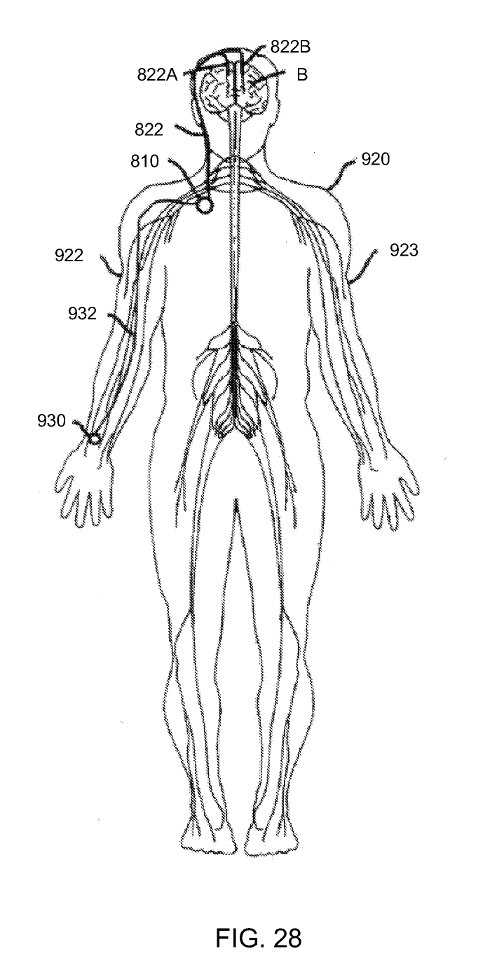

Referring to FIG. 28, a therapy delivery device 810 is implanted in a human body 920 in the location shown or may be implanted in any other suitable location. Body 920 includes arms 922 and 923. In various embodiments and as depicted, catheter 822 is divided into twin or similar tubes 822A and 822B that are implanted into the brain bilaterally. In some embodiments, tube 822B is supplied with a composition from a separate catheter and pump. Of course, unilateral delivery may be performed in accordance with the teachings presented herein.

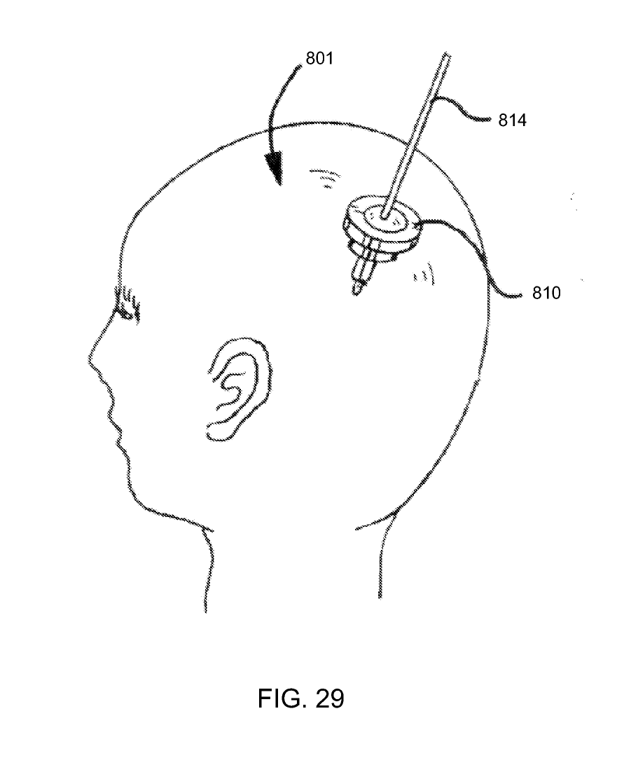

Referring to FIG. 29, a composition including a therapeutic agent may be delivered to a subject's CNS via an injection port 810 implanted subcutaneously in the scalp of a patient 801, e.g. as described in U.S. Pat. No. 5,954,687 or otherwise known in the art. A guide catheter 814 may be used to guide an infusion catheter through port 810 to a target location. Of course, an infusion catheter may be directly be inserted through port 810 to the target location.

Further examples of suitable catheters and delivery systems for administering an agent to the brain (intraparenchymally or intracerebroventricularly) are described in WO 2008/054700, entitles INFUSION CATHETERS, published on May 8, 2008, and assigned to Medtronic, Inc. Of course, any other known or developed implantable or external infusion device, port, or the like may be employed to deliver agents directly to a subject's CNS.

One or more therapeutic agent may be administered directly to the CNS of a subject for purposes of treatment or study; e.g. as discussed above. For example, a therapeutic agent may be administered to study the effects of the agent on a subject at a molecular level, a pharmacological level, at a physiological level, at a behavioral level, or the like. By way of further example, any one or more condition, symptom, or disease may be treated, e.g. as discussed above. For example, the diseases such as Alzheimer's disease (AD), cerebral amyloid angiopathy (CAA), Lewy body dementia, Down's syndrome (DS), and Parkinson's disease (PD) may be treated or studied.

Any suitable therapeutic agent may be administered for purposes of investigation or treatment; e.g. as discussed above. Therapeutic agents can be administered in the form of pharmaceutical compositions; e.g. as discussed above.

In various embodiments, the compositions are formulated as injectable compositions; e.g. as discussed above.

A therapeutic agent may be administered at any suitable dos; e.g. as discussed above.

Administration of one or more therapeutic agents directly to the CNS of a subject may be performed to treat or study a variety of disease states; e.g. as discussed. In various embodiments, agents described herein are used to treat or study a disease associated with increased or aberrant soluble Aβ, amyloid fibrils or amyloid plaques. Examples of disease associated with increased or aberrant soluble Aβ, amyloid fibrils or amyloid plaques include Alzheimer's disease (AD), cerebral amyloid angiopathy (CAA), Lewy body dementia, and Down's syndrome (DS); as discussed above.

In various embodiments, a method includes identifying a subject suffering from or at risk of one or more of such diseases; e.g., as discussed above, and administering one or more therapeutic agents directly to the subject's CNS.

In various embodiments, cerebral plaques may be cleared or prevented from forming by administering one or more therapeutic agents directly to the subject's CNS. The methods may further include clearing or preventing parenchymal amyloid plaques or soluble forms of Aβ; e.g. as discussed above. The methods may further include improving cognitive aspects of the subject; e.g., as discussed above.

In some embodiments, cognitive abilities of a subject are improved by administering one or more therapeutic agents directly to the subject's CNS.

In various embodiments, parenchymal amyloid plaques or soluble forms of Aβ are cleared in a subject by administering one or more therapeutic agents directly to the subject's CNS.

The ability of a therapy described herein to treat a disease may be evaluated through medical examination, or by diagnostic or other tests, e.g. as discussed above.

Thus, embodiments of APHERESIS, ADMINISTRATION OF AGENT OR COMBINATION THEREOF are disclosed. One skilled in the art will appreciate that the present invention can be practiced with embodiments other than those disclosed. The disclosed embodiments are presented for purposes of illustration and not limitation.

What is claimed is:

1. A method comprising:
withdrawing fluid from a first cerebrospinal fluid compartment of a subject;
passing the withdrawn fluid through a reservoir of a medical device to remove the target molecule from the withdrawn fluid, wherein the reservoir contains a medium comprising a solid support to which an antibody directed to the target molecule is bound and wherein the media is capable of removing the target molecule from the cerebrospinal fluid;
adding a therapeutic agent to the withdrawn fluid with removed target molecule; and
delivering the withdrawn fluid with removed target molecule and added therapeutic agent to a second cerebrospinal fluid compartment of the subject,
wherein the first and second cerebrospinal fluid compartments are the same or different.

2. The method of claim 1, wherein the target molecule is amyloid beta.

3. The method of claim 1, wherein the therapeutic agent is selected from the group consisting of an ApoE-modulating agent; a RAGE inhibitor; a β-secretase 1 (BACE1) inhibitor; a γ-secretase inhibitor; a muscarinic receptor subtype 1 (M1) agonists; a growth factor; an enzyme capable of degrading amyloid beta; a mitochondrial antioxidant; insulin; and an inhibitor of tumor necrosis factor (TNF).

4. The method of claim 1, wherein the therapeutic agent is a β-secretase 1 (BACE1) inhibitor.

5. A method for treating a disease associated with amyloid beta accumulation in a subject in need thereof, comprising:
delivering directly to the central nervous system of the subject a therapeutic agent selected from the group consisting of an ApoE-modulating agent; a RAGE inhibitor; a β-secretase 1 (BACE1) inhibitor; a γ-secretase inhibitor; a muscarinic receptor subtype 1 (M1) agonist; a growth factor; an enzyme capable of degrading amyloid beta; a mitochondrial antioxidant; insulin; and an inhibitor of tumor necrosis factor (TNF).

6. The method of claim 5, further comprising delivering an anti-amyloid beta antibody to the subject.

7. The method of claim 5, wherein the therapeutic agent is a liver X receptor (LXR) agonist.

8. The method of claim 5, wherein the therapeutic agent is the RAGE inhibitor, PF-04494700.

9. The method of claim 5, wherein the therapeutic agent is a γ-secretase inhibitor selected from the group consisting of r-flurbiprofen, MCP-7869, LY-450139, LY411575, and MK0752.

10. The method of claim 5, wherein the therapeutic agent is an M1 agonist selected from the group consisting of cevimeline, talsaclidine, sabcomeline and milameline, xanomeline, and 5-(3-ethyl-1,2,4-oxadiazol-5-yl)-1,4,5,6-tetrahydropyrimmidine (CDD-0102).

11. The method of claim 5, wherein, wherein the therapeutic agent is a growth factor selected from the group consisting of VEGF, BDNF, NGF, and IGF-1.

12. The method of claim 5, wherein the therapeutic agent is an enzyme capable of degrading amyloid beta selected from the group consisting of neprilysin, insulin-degrading enzyme (IDE), angiotensin-converting enzyme (ACE), and insulysin.

13. The method of claim 5, wherein the therapeutic agent is the microtubule stabilizing agent, NAP (AL-108).

14. The method of claim 5, wherein the therapeutic agent is a β-secretase 1 (BACE1) inhibitor.

15. The method of claim 5, wherein the therapeutic agent is insulin.

16. The method of claim 5, wherein the therapeutic agent is an inhibitor of tumor necrosis factor.

17. The method of claim 5, wherein the therapeutic agent is administered to cerebrospinal fluid of the subject.

18. The method of claim 5, wherein the therapeutic agent is administered intraparenchymally.

19. The method of claim 5, wherein the therapeutic agent is administered to a hippocampus of the subject.
